(19) United States
(12) Patent Application Publication
(10) Pub. No.: US 2011/0015458 A1
(43) Pub. Date: Jan. 20, 2011

(54) CONVERSION OF HYDROGEN BROMIDE TO ELEMENTAL BROMINE

(75) Inventors: John J. Waycuilis, Cypress, TX (US); Patrick K. Moore, Lake Jackson, TX (US); Greg A. Lisewsky, Seabrook, TX (US)

Correspondence Address: MARATHON OIL COMPANY;C/O LAW OFFICE OF JACK E. EBEL, 165 SOUTH UNION BOULEVARD, SUITE 902, LAKEWOOD, CO, 80228, US

(73) Assignee: Marathon GTF Technology, Ltd., Houston, TX (US)

(21) Appl. No.: 12/792,335

(22) Filed: Jun. 2, 2010

Related U.S. Application Data
(60) Provisional application No. 61/225,915, filed on Jul. 15, 2009.

(51) Int. Cl. C07C 2/00; C01B 7/09

(52) U.S. Cl. 585/310; 423/502

(57) ABSTRACT

A method is provided for converting hydrogen bromide to elemental bromine. A portion of an initial hydrogen bromide-rich gas is thermally oxidized at a thermal oxidation temperature to produce a first fraction of elemental bromine and a remainder of the initial hydrogen bromide-rich gas. At least a portion of the remainder of the initial hydrogen bromide-rich gas is catalytically oxidized at a lower catalytic oxidation temperature to produce a second fraction of elemental bromine.

### BACKGROUND OF THE INVENTION

The present invention relates to the conversion of hydrogen bromide to elemental bromine and, more particularly, in one or more embodiments, to a method, wherein gaseous hydrogen bromide is converted to elemental bromine via a thermal oxidation stage and a catalytic oxidation stage.

Conventional industrial processes exist, wherein a feedstock is brominated using elemental bromine to produce reactive bromide intermediates. The reactive bromide intermediates are in turn utilized for the synthesis of valuable end products. These synthesis reactions typically produce hydrogen bromide as a byproduct which is frequently discharged to the environment as a waste stream. However, the hydrogen bromide byproduct is not environmentally compatible so in most cases it must first be neutralized before it is discharged to the environment in order to meet environmental regulatory standards.

Since elemental bromine is a relatively valuable reagent and there are attendant costs associated with neutralization and discharge of hydrogen bromide, a preferred alternative is to recover the hydrogen bromide from the effluent of the synthesis reactor, convert it back to elemental bromine, and return it to the bromination reactor as a recycle stream. This alternative is currently practiced in existing processes, for example, U.S. Pat. Nos. 7,244,876 and 7,348,464 both to Waycuilis, which are incorporated herein by reference. Both references disclose a bromination/synthesis process for the conversion of gaseous alkanes to liquid hydrocarbons. Gaseous hydrogen bromide is produced as a byproduct of the synthesis reaction. The process recovers the hydrogen bromide downstream and contacts it with water to form a fully ionized aqueous hydrogen bromide liquid. The resulting liquid is neutralized and converted to elemental bromine, which is recycled back upstream to the bromination reaction.

A need exists for alternate methods for converting hydrogen bromide to elemental bromine which exhibit improved efficiency and cost effectiveness over those methods presently known and practiced. The present invention as described hereafter is directed toward satisfying this need.

### SUMMARY OF THE INVENTION

The present invention is a method for converting hydrogen bromide to elemental bromine. A portion of an initial hydrogen bromide-rich gas is thermally oxidized at a thermal oxidation temperature to produce a first fraction of elemental bromine and a remainder of the initial hydrogen bromide-rich gas. At least a portion of the remainder of the initial hydrogen bromide-rich gas is catalytically oxidized at a catalytic oxidation temperature to produce a second fraction of elemental bromine.

A preferred initial hydrogen bromide-rich gas is a substantially dry gas mixture. A preferred thermal oxidation temperature is substantially greater than the catalytic oxidation temperature, which is preferably in a range of about 250° C. to about 345° C. Thermal oxidation of the portion of the initial hydrogen bromide-rich gas preferably converts about 80% to 99%, and more preferably about 85% to 95%, of total hydrogen bromide in the initial hydrogen bromide-rich gas to elemental bromine. Catalytic oxidation of the at least a portion of the remainder of the initial hydrogen bromide-rich gas preferably converts about 20% to 1%, and more preferably about 15% to 5% of total hydrogen bromide in the initial hydrogen bromide-rich gas to elemental bromine.

In accordance with one embodiment, the present method further comprises adding an oxidizing gas, preferably pure oxygen or a gas mixture containing oxygen such as air, to the initial hydrogen bromide-rich gas while or before thermally oxidizing the hydrogen bromide-rich gas. In accordance with another embodiment, the present method further comprises deriving the initial hydrogen bromide-rich gas from a hydrogen bromide-containing gas. In accordance with another embodiment, the hydrogen bromide-containing gas has a lower hydrogen bromide concentration than the initial hydrogen bromide-rich gas. The hydrogen bromide-containing gas is, in another alternative, a gaseous mixture containing hydrogen bromide and lower molecular weight hydrocarbons. Alternatively, the initial hydrogen bromide-rich gas and the hydrogen bromide-containing gas are the same.

A preferred hydrogen bromide-containing gas is derived from an upstream process. The upstream process is either an associated process or an unrelated process. A preferred upstream process is a gaseous alkane conversion process, wherein gaseous alkanes are converted to liquid hydrocarbons by brominating the gaseous alkanes and catalytically reacting the resulting alkyl bromides to form the liquid hydrocarbons and hydrogen bromide.

In accordance with yet another embodiment, the present method further comprises converting gaseous alkanes to liquid hydrocarbons in a gaseous alkane conversion process by brominating the gaseous alkanes and catalytically reacting the resulting alkyl bromides to form the liquid hydrocarbons and a hydrogen bromide-containing gas. The initial hydrogen bromide-rich gas is derived from the hydrogen bromide-containing gas. In accordance with still another embodiment, the present method further comprises recycling the first and second fractions of elemental bromine as a feed to the process for converting gaseous alkanes to liquid hydrocarbons by brominating the gaseous alkanes and catalytically reacting the resulting alkyl bromides to form liquid hydrocarbons and hydrogen bromide.

The present invention is alternately characterized as a method for converting hydrogen bromide to elemental bromine by adding an oxidizing gas to an initial hydrogen bromide-rich gas to form a thermal oxidation feed gas. The initial hydrogen bromide-rich gas is a substantially dry gas mixture including hydrogen bromide. A portion of the thermal oxidation feed gas is thermally oxidized in a thermal oxidation reactor at a thermal oxidation temperature to produce a first fraction of elemental bromine and a remainder of the thermal oxidation feed gas. At least a portion of the remainder of the thermal oxidation feed gas is catalytically oxidized in a catalytic reactor at a catalytic oxidation temperature to produce a second fraction of elemental bromine. The thermal oxidation temperature is substantially greater than the catalytic oxidation temperature.

In accordance with one embodiment, the method further comprises recovering the first and second fractions of elemental bromine as an elemental bromine product from a catalytic reactor effluent gas discharged from the catalytic reactor. In accordance with another embodiment, the method further comprises condensing the catalytic reactor effluent gas to obtain a three-phase mixture comprising a gas phase, an elemental bromine liquid phase, and an aqueous liquid phase. In accordance with yet another embodiment, the method further comprises separating the gas phase, the elemental bromine liquid phase, and the aqueous liquid phase from one another. The elemental bromine liquid phase is essentially pure elemental bromine in a liquid state and comprises a first portion of the elemental bromine product.

In accordance with still another embodiment, the gas phase includes oxygen and a first residual elemental bromine portion. The method further comprises separating recovering the first residual elemental bromine as a second portion of the elemental bromine product. In accordance with still another embodiment, the aqueous liquid phase includes water and a second residual elemental bromine portion dissolved therein. The method further comprises recovering the second residual elemental bromine as a third portion of the elemental bromine product.

The present invention is alternately characterized as a method for converting hydrogen bromide to elemental bromine by converting gaseous alkanes to liquid hydrocarbons in a gaseous alkane conversion process by brominating the gaseous alkanes and catalytically reacting the resulting alkyl bromides to form the liquid hydrocarbons and a hydrogen bromide-containing gas. An initial hydrogen bromide-rich gas is derived from the hydrogen bromide-containing gas. A portion of the initial hydrogen bromide-rich gas is thermally oxidized at a thermal oxidation temperature to produce a first fraction of elemental bromine and a remainder of the initial hydrogen bromide-rich gas. At least a portion of the remainder of the initial hydrogen bromide-rich gas is catalytically oxidized at a catalytic oxidation temperature to produce a second fraction of elemental bromine. The first and second fractions of elemental bromine are recycled to the gaseous alkane conversion process to brominate the gaseous alkanes.

In accordance with one embodiment, the hydrogen bromide-containing gas has a lower hydrogen bromide concentration than the initial hydrogen bromide-rich gas. The hydrogen bromide-containing gas is, in another alternative, a gaseous mixture containing hydrogen bromide and lower molecular weight hydrocarbons. Alternatively, the initial hydrogen bromide-rich gas and the hydrogen bromide-containing gas are the same.

The invention will be further understood from the accompanying drawings and description.

### BRIEF DESCRIPTION OF THE DRAWINGS

The accompanying drawings illustrate certain aspects of the present invention, but should not be viewed as by themselves limiting or defining the invention.

FIG. 1 is a simplified block diagram of the method of the present invention which conceptually divides the method into a sequence of process stages;

FIG. 2 is a schematic view of a system employed in the practice of an embodiment of the present method, wherein the hydrogen bromide recovery stage of the method operates in a circulating regeneration mode;

FIG. 3 is a schematic view of a system employed in the practice of an alternate embodiment of the present method, wherein the hydrogen bromide recovery stage of the method operates in a swing regeneration mode;

FIG. 4 is a graphical depiction of a thermodynamic equilibrium calculation versus temperature for the thermal oxidation reaction of gaseous hydrogen bromide with excess air;

FIG. 5 is a graphical depiction of hydrogen bromide thermal oxidation conversion versus temperature in the thermal oxidation reactor zone in accordance with the method of Example 1;

FIG. 6 is a graphical depiction of hydrogen bromide thermal oxidation conversion versus the amount of excess air fed to the thermal oxidation unit in accordance with the method of Example 2;

FIG. 7 is a schematic view of an embodiment of the hydrogen bromide conversion method of the present invention in the form of a process flow diagram which is practiced in accordance with Example 4;

FIG. 8 is a simplified block flow diagram of an embodiment of the process of the present invention;

FIG. 9 is a schematic view of one embodiment of the process of the present invention;

FIG. 10 is a schematic view of another embodiment of process of the present invention;

FIG. 11A is schematic view of another embodiment of the process of the present invention;

FIG. 11B is a schematic view of the embodiment of the process of the present invention illustrated in FIG. 11A depicting an alternative processing scheme which may be employed when oxygen is used in lieu of air in the oxidation stage;

FIG. 12A is a schematic view of the embodiment of the process of the present invention illustrated in FIG. 11A with the flow through the metal oxide beds being reversed;

FIG. 12B is a schematic view of the embodiment of the process of the present invention illustrated in FIG. 12A depicting an alternative processing scheme which may be employed when oxygen is used in lieu of air in the oxidation stage;

FIG. 13A is a schematic view of another embodiment of the process of the present invention;

FIG. 13B is a schematic view of the embodiment of the process of the present invention illustrated in FIG. 13A depicting an alternative processing scheme which may be employed when oxygen is used in lieu of air in the oxidation stage;

FIG. 14 is a schematic view of another embodiment of the process of the present invention;

FIG. 15 is a schematic view of the embodiment of the process of the present invention illustrated in FIG. 14 with the flow through the metal oxide beds being reversed;

FIG. 16 is a schematic view of another embodiment of the process of the present invention;

FIG. 17 is a graph of methyl bromide conversion and product selectivity for the oligomerization reaction of the process of the present invention as a function of temperature;

FIG. 18 is a graph comparing conversion and selectivity for the example of methyl bromide, dry hydrobromic acid and methane versus only methyl bromide plus methane;

FIG. 19 is a graph of product selectivity from reaction of methyl bromide and dibromomethane vs. product selectivity from reaction of methyl bromide only;

FIG. 20 is a graph of a Paraffinic Olefinic Napthenic and Aromatic (PONA) analysis of a typical condensed product sample of the process of the present invention; and

FIG. 21 is a graph of a PONA analysis of another typical condensed product sample of the present invention.

### DESCRIPTION OF PREFERRED EMBODIMENTS

The present invention relates to the conversion of gaseous hydrogen bromide (HBr) to elemental bromine (Br₂) and, more particularly, in one or more embodiments, to a method, wherein gaseous hydrogen bromide is converted to elemental bromine via a sequential thermal oxidation stage and a catalytic oxidation stage.

Certain embodiments of the method of the present invention are described below. Although aspects of what is to believed to be the primary chemical reaction involved in the present invention are discussed as it is believed to occur, it should be understood that side reactions may take place. One should not assume that the failure to discuss any particular side reaction herein means that this side reaction does not occur. Conversely, the primary reaction discussed below should not be considered exhaustive or limiting.

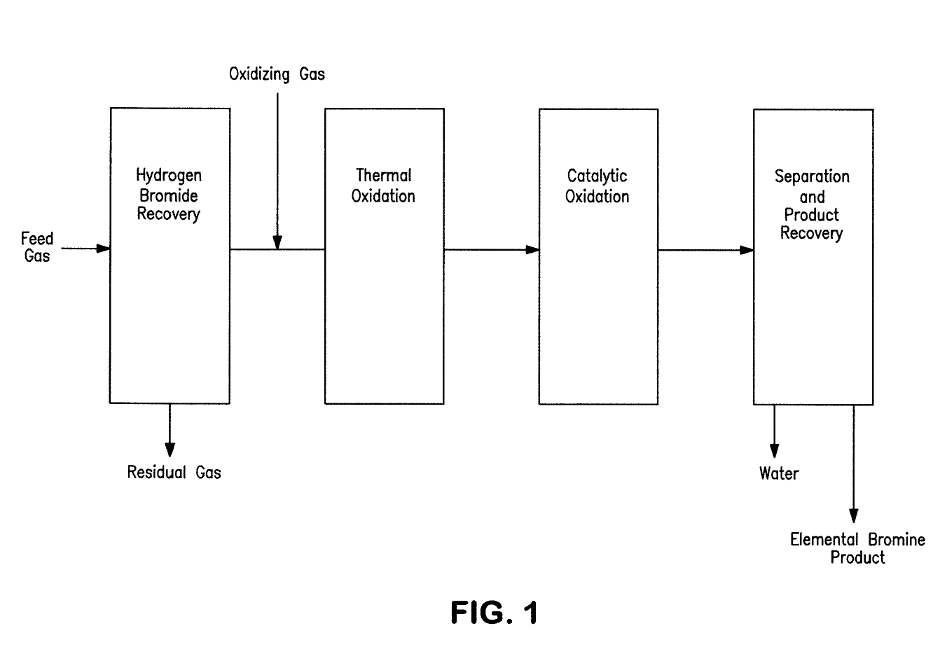

FIG. 1 conceptually depicts the method of the present invention as divided into a sequence of stages. In accordance with this general depiction, a feed gas is provided which is a hydrogen bromide-containing gas containing hydrogen bromide and optionally one or more other constituents. Where one or more other constituents are present along with the hydrogen bromide in a gas mixture, the gas mixture is preferably pretreated in an optional hydrogen bromide recovery stage to separate and recover an initial hydrogen bromide-rich gas from the other constituents of the gas mixture.

The remaining constituents may be discharged as a residual gas if desired. The initial hydrogen bromide-rich gas is mixed with an oxidizing gas and heated in the thermal oxidation stage. Portions of the hydrogen bromide-rich gas are oxidized at high temperature in the thermal oxidation stage to produce elemental bromine and steam.

The unreacted remainder of the hydrogen bromide-rich gas and oxidizing gas is conveyed from the thermal oxidation stage to the catalytic oxidation stage where most or substantially all of the remaining unreacted hydrogen bromide-rich gas is oxidized in the presence of a catalyst to produce additional elemental bromine and steam. The resulting mixture of elemental bromine and steam is fed to a separation and product recovery stage where the steam is condensed to water. The resulting water and elemental bromine are separated and the elemental bromine is recovered as the end product.

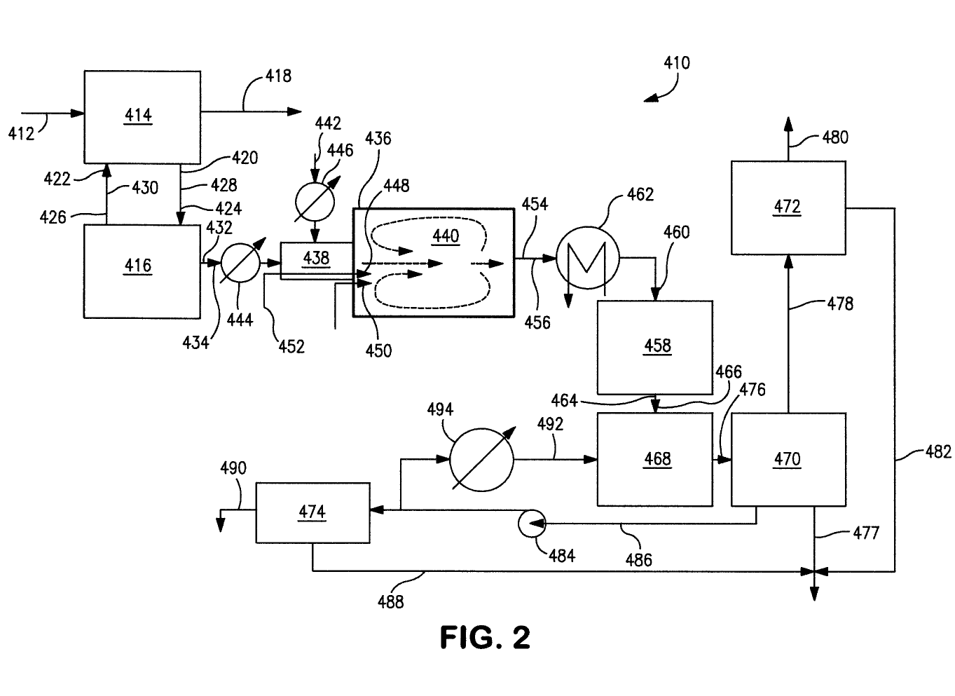

A specific embodiment of the method of the present invention is described in further detail with reference to FIG. 2. The method of this embodiment is practiced using a system of process equipment shown schematically in FIG. 2 and generally designated by the reference character 410.

The system 410 has a feed gas line 412 at its upstream end which is preferably a pipe, conduit, or the like for introducing a feed gas into the system 410. Substantially any gas which contains hydrogen bromide, i.e., hydrogen bromide-containing gas, has utility as the feed gas of the present invention. The feed gas can be an essentially pure hydrogen bromide gas or a gas mixture containing hydrogen bromide and one or more other constituents, although the feed gas is preferably a dry gas. In practice, a gas mixture rather than a pure gas is usually more readily available as the hydrogen bromide-containing gas for the feed gas.

A gas mixture used as the feed gas preferably contains at least about 20 mol % hydrogen bromide but more preferably about 33 to 50 mol % hydrogen bromide. Examples of the one or more other constituents which can be present in the gas mixture include methane and other light alkanes, alkyl bromides and any combination thereof. However, it is understood that the above-recited list of constituents is merely exemplary and is not limiting to the number or type of constituents present in the gas mixture. Nevertheless, water vapor is preferably excluded as a constituent of the gas mixture. In other words, the gas mixture is preferably characterized as a dry gas, i.e., the gas mixture preferably contains less than about 10 mol % water vapor.

The system 410 additionally includes a hydrogen bromide separation unit 414 and a separation medium regeneration unit 416, which in combination constitute an embodiment of the hydrogen bromide recovery stage shown conceptually in FIG. 1. The specific cooperative interconnection of the hydrogen bromide separation unit 414 and the separation medium regeneration unit 416 to one another as shown in FIG. 2 constitutes an embodiment of the system 410 which enables operation of the hydrogen bromide recovery stage in a circulating regeneration mode as described hereafter.

In accordance with the present embodiment, the feed gas is conveyed via the feed gas line 412 to the hydrogen bromide separation unit 414, which is an enclosed vessel, chamber, container, or the like containing a liquid absorbent (i.e., a liquid solvent) or a solid adsorbent. The absorbent or adsorbent is relatively selective for the absorption or adsorption of hydrogen bromide to the exclusion of the other constituents in the gas mixture. In addition to appropriate absorption or adsorption selectivity, it is further desirable that the hydrogen bromide-loaded absorbent or adsorbent is regenerable in a practical manner to enable desorption of the hydrogen bromide therefrom. Essentially any absorbent or adsorbent known to a skilled artisan satisfying these criteria can have utility in the present method.

A preferred liquid absorbent satisfying the above-recited criteria is the non-aqueous solvent, N-Methyl-2-pyrrolidone (NMP). Alternate liquid absorbents include aqueous solvents, such as azeotropic hydrobromic acid (about 48 wt. %), and non-aqueous polar or non-polar aprotic or ionic solvents such as liquid amines, ethers, and glycols, including polyethylene glycols and, more particularly, methyl-ether-polyethylene glycol. A preferred solid adsorbent satisfying the above-recited criteria is silica gel. Alternate solid adsorbents include zeolites, solid polymeric amines, solid high molecular weight polyethylene glycols and ion exchange resins. The term “separation medium” is used in some instances hereafter and is inclusive of both liquid absorbents and solid adsorbents.

The operating conditions of the hydrogen bromide separation unit 414, including pressure and temperature, are a function, at least in part, of the particular separation medium selected. Nevertheless, the hydrogen bromide separation unit 414 is typically operated at a pressure in the range of about 1 bar to about 40 bar and a temperature in a range of about −50° C. to about 70° C. As can be appreciated by a skilled artisan, the gas feed rate to the hydrogen bromide separation unit 414 is likewise a function, at least in part, of the selected separation medium, the operating pressure and temperature of the unit 414, and the size and geometry of the unit 414.

The hydrogen bromide separation unit 414 functions to separate the hydrogen bromide from the gas mixture by means of the separation medium, thereby producing a hydrogen bromide-rich gas and a residual gas. The hydrogen bromide-rich gas is the portion of the gas mixture which is absorbed or adsorbed by the separation medium in the hydrogen bromide separation unit 414. Conversely, the residual gas is the remaining portion of the gas mixture which has not been absorbed or adsorbed by the separation medium in the hydrogen bromide separation unit 414.

Due to the selectivity of the separation medium, the hydrogen bromide-rich gas absorbed or adsorbed by the separation medium is preferably comprised mostly of hydrogen bromide, i.e., the hydrogen bromide-rich gas preferably contains at least about 90 mol % hydrogen bromide. More preferably the hydrogen bromide-rich gas consists essentially entirely of hydrogen bromide with the exception of trace amounts of other constituents, i.e., the hydrogen bromide-rich gas more preferably contains at least about 99 mol % hydrogen bromide. As such, the hydrogen bromide-rich gas is preferably a dry gas which is substantially free of water, i.e., the hydrogen bromide-rich gas preferably contains less than about 10 mol % water vapor.

Due to the separation efficiency of the separation medium, most of the hydrogen bromide in the feed gas entering the hydrogen bromide separation unit 414 is preferably absorbed or adsorbed by the separation medium as the hydrogen bromide-rich gas, i.e., the hydrogen bromide-rich gas preferably contains at least about 90 mol % of the hydrogen bromide from the feed gas. More preferably essentially the entirety of the hydrogen bromide in the feed gas is absorbed or adsorbed by the separation medium as the hydrogen bromide-rich gas, i.e., the hydrogen bromide-rich gas more preferably contains at least about 99 mol % of the hydrogen bromide from the feed gas. As such, the amount of hydrogen bromide remaining in the residual gas is relatively low or even negligible, i.e., the residual gas preferably contains no more than about 1 mol % hydrogen bromide.

The hydrogen bromide separation unit 414 is provided with a residual gas outlet line 418 which enables discharge of the residual gas produced in the hydrogen bromide separation unit 414 from the system 410. The discharged residual gas can be disposed in an environmentally acceptable manner or recovered for further processing and/or applications outside the system 410. For example, the residual gas can be recycled to the feed of an associated bromination reactor (not shown) upstream of the system 410.

The separation medium, although preferably highly selective and efficient, has a finite capacity for absorption or adsorption of the hydrogen bromide. Therefore, in order to operate the hydrogen bromide separation unit 414 in a practical manner, it is desirable to regenerate the separation medium at or before the point where its hydrogen bromide loading approaches capacity (i.e., saturation) and/or at or before the point where the separation medium otherwise exhibits diminished hydrogen bromide loading capability. Accordingly, the separation medium regeneration unit 416, which is preferably an enclosed vessel, chamber, container, or the like, such as a fractionation column, generally functions to regenerate the loaded separation medium to an unloaded state.

The separation medium regeneration unit 416 desorbs most or essentially all of the hydrogen bromide-rich gas from the loaded separation medium by conventional pressure or thermal means, which do not substantially degrade the separation medium. As a result, desorption frees the hydrogen bromide-rich gas from the separation medium while simultaneously restoring the separation medium to its substantially unloaded state. The operating conditions of the separation medium regeneration unit 416 are a function, at least in part, of the particular separation medium selected. Nevertheless, where pressure is the primary desorption mechanism, the separation medium regeneration unit 416 is typically operated at a pressure in a range of about 0.1 bar to about 10 bar and a temperature in a range of about 0° C. to about 70° C. Where heat is the primary desorption mechanism, the separation medium regeneration unit 416 is typically operated at a pressure in a range of about 1 bar to about 40 bar and a temperature in a range of about 50° C. to about 300° C.

As noted above, the cooperative interconnection of the hydrogen bromide separation unit 414 and separation medium regeneration unit 416 shown in FIG. 2 and described as follows enables operation of the hydrogen bromide recovery stage in the circulating regeneration mode. In particular, the hydrogen bromide separation unit 414 has a loaded separation medium outlet port 420 and a regenerated separation medium inlet port 422. The separation medium regeneration unit 416 similarly has a loaded separation medium inlet port 424 and a regenerated separation medium outlet port 426. A loaded separation medium line 428 extends between the loaded separation medium outlet port 420 of the hydrogen bromide separation unit 414 and the loaded separation medium inlet port 424 of the separation medium regeneration unit 416 to provide communication therebetween. A regenerated separation medium line 430 extends between the regenerated separation medium inlet port 422 of the hydrogen bromide separation unit 414 and the regenerated separation medium outlet port 426 of the separation medium regeneration unit 416 to likewise provide communication therebetween.

The separation medium regeneration unit 416 additionally has a hydrogen bromide-rich gas outlet port 432. A hydrogen bromide-rich gas line 434 extends from the hydrogen bromide-rich gas outlet port 432 to the thermal oxidation stage shown conceptually in FIG. 1 and described in more detail below in the context of the system 410.

During operation of the hydrogen bromide recovery stage in the circulating regeneration mode, the separation medium is continuously circulated in a closed loop between the hydrogen bromide separation unit 414 and the separation medium regeneration unit 416. A single pass through the loop comprises withdrawing the loaded separation medium from the hydrogen bromide separation unit 414 and conveying it to the separation medium regeneration unit 416 via the loaded separation medium outlet port 420 and loaded separation medium line 428. The loaded separation medium is introduced into the separation medium regeneration unit 416 via the loaded separation medium inlet port 424 and the hydrogen bromide-rich gas is desorbed from the loaded separation medium therein to regenerate the separation medium. As the hydrogen bromide-rich gas is desorbed from the loaded separation medium, the resulting freed hydrogen bromide-rich gas is conveyed to the thermal oxidation stage via the hydrogen bromide-rich gas outlet port 432 and the hydrogen bromide-rich gas line 434.

After regeneration, the regenerated separation medium is withdrawn from the separation medium regeneration unit 416 and conveyed to the hydrogen bromide separation unit 414 via the regenerated separation medium outlet port 426 and regenerated separation medium line 430. The regenerated separation medium is introduced into the hydrogen bromide separation unit 414 via the regenerated separation medium inlet port 422 therein, thereby completing one pass of the separation medium through the loop. The separation medium makes additional passes through the loop in a continuous manner as long as the system 410 remains in operation. It is noted that the circulating regeneration mode is applicable to both liquid and solid separation media.

The hydrogen bromide recovery stage is operated in the circulating regeneration mode in a manner which preferably sets the residence time of the separation medium in the hydrogen bromide separation unit 414 at a value sufficient to enable substantial loading of the hydrogen bromide-rich gas on the separation medium. The residence time of the separation medium in the separation medium regeneration unit 416 is likewise preferably sufficient to enable substantial desorption of the hydrogen bromide-rich gas from the separation medium. This results in optimal utilization of the separation medium.

The hydrogen bromide separation unit 414 and separation medium regeneration unit 416 are each shown in FIG. 2 and described above for purposes of illustration as being single vessels. However, it is understood that the present invention is not limited in this manner. Although not shown, it is within the purview of a skilled artisan and within the scope of the present invention to employ an interconnected network of multiple vessels as the hydrogen bromide separation unit and/or as the separation medium regeneration unit to increase the capacity and/or efficiency of the hydrogen bromide recovery stage.

The vessels of a multiple-vessel unit may operate in series, such as an in the case of multi-stage countercurrent contact of the separation medium with the hydrogen bromide containing gas. Alternatively, the vessels may operate in parallel in cooperation with one another. For example, multiple smaller vessels may be utilized in parallel to obtain larger capacity. In particular, the parallel vessels may be linked together to give a larger total capacity for separation unit 414 and/or regeneration unit 416 or pairs of separation unit and regeneration unit vessels may be operated independently, but in parallel, to give larger overall capacity. In any case, the individual operation of each vessel is substantially the same as described above with respect to the single-vessel unit and achieves substantially the same result as is apparent to a skilled artisan.

Conversely, although likewise not shown, it is within the purview of a skilled artisan and within the scope of the present invention to integrate both the hydrogen bromide separation unit and separation medium regeneration unit into a single vessel having multiple chambers or zones included therein which enable the distinct steps of hydrogen bromide-rich gas absorption/adsorption and separation medium regeneration to be practiced within the same vessel.

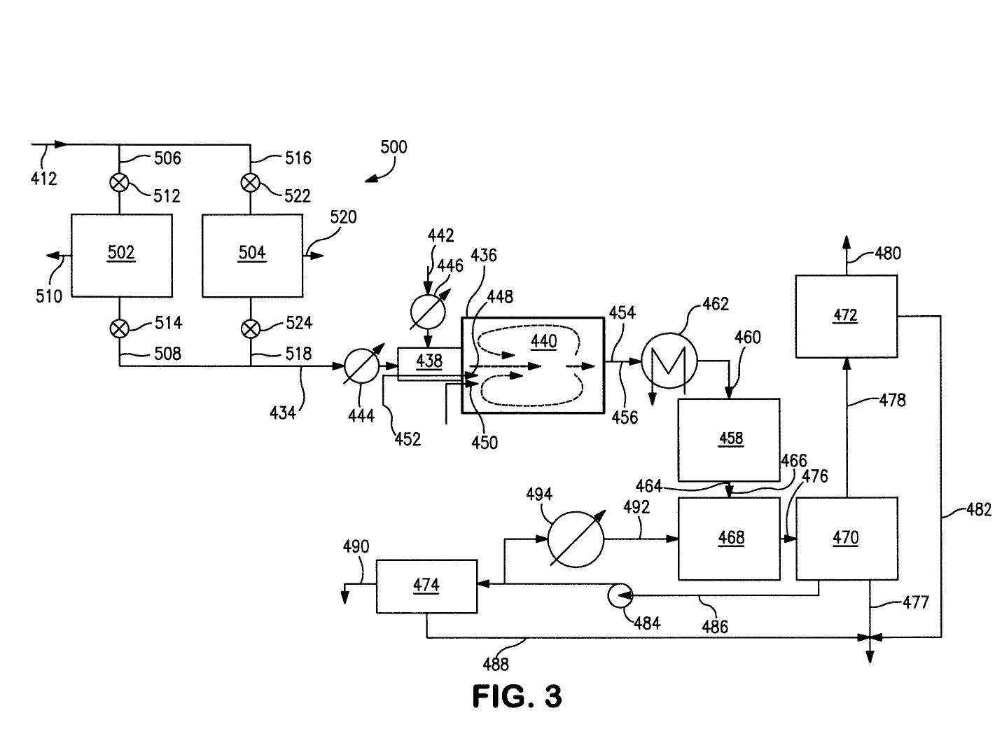

An alternate embodiment of the present method enables operation of the hydrogen bromide recovery stage in a swing regeneration mode. The method of this embodiment is practiced using a system of process equipment shown schematically in FIG. 3 and generally designated by the reference character 500. The system 500 of FIG. 3 differs from the system 410 of FIG. 2 only in the structural elements of the hydrogen bromide recovery stage and their attendant operation. The remaining elements of the system 500 shown in FIG. 3 are common to the system 410 shown in FIG. 2 and are designated in FIG. 3 by the same reference characters as used in FIG. 2.

The system 500 comprises a first dual-function unit 502 and a second dual-function unit 504 which in combination constitute an alternate embodiment of the hydrogen bromide recovery stage shown conceptually in FIG. 1. Each unit 502, 504 is essentially identical to the other. As such, each unit 502, 504 is an enclosed vessel, chamber, container, or the like containing a selective and regenerable solid adsorbent such as the preferred adsorbent recited above, silica gel. It is noted that although it may be possible to operate the system 500 in the swing regeneration mode using liquid separation media, it is more preferable to operate in the swing regeneration mode using a solid separation medium. This distinguishes the swing regeneration mode from the circulating regeneration mode which exhibits no preference between solid and liquid media.

The specific cooperative interconnection of the first dual-function unit 502 and the second dual-function unit 504 to one another as shown in FIG. 3 enables operation of the units 502, 504 in a parallel cyclical manner described below which is the foundation of the swing regeneration mode. In particular, the first dual-function unit 502 has a first feed gas inlet line 506, a first hydrogen bromide-rich gas outlet line 508 and a first residual gas outlet line 510. In-line valves 512, 514 are positioned in the first feed gas inlet line 506 and the first hydrogen bromide-rich gas outlet line 508, respectively, enabling an operator of the system 500 to selectively regulate gas flow therethrough in a manner described below. The second dual-function unit 504 similarly has a second feed gas inlet line 516, a second hydrogen bromide-rich gas outlet line 518 and a second residual gas outlet line 520. In-line valves 522, 524 are positioned in the second feed gas inlet line 516 and the second hydrogen bromide-rich gas outlet line 518, respectively, enabling an operator of the system 500 to selectively regulate gas flow therethrough in a manner described below.

During operation of the hydrogen bromide recovery stage in the swing regeneration mode, each dual-function unit 502, 504 cycles in parallel over time so that each unit 502, 504 alternately provides a hydrogen bromide separation function and a separation medium regeneration function. Accordingly, when the first dual-function unit 502 operates to provide the hydrogen bromide separation function, the second dual-function unit 504 operates to provide the separation medium regeneration function. Conversely, when the second dual-function unit 504 operates to provide the hydrogen bromide separation function, the first dual-function unit 502 operates to provide the separation medium regeneration function.

It is noted that, unlike the circulating regeneration mode of operation, the separation medium of each dual-function unit 502, 504 is retained within its respective unit as long as the system 500 remains in the swing mode of operation. Nevertheless, when the first dual-function unit 502 (or the second dual-function unit 504 when switched over) is operating in the hydrogen bromide separation function, its operating parameters and resulting output are essentially the same as those of the hydrogen bromide separation unit 414 described above. Likewise, when the second dual-function unit 504 (or the first dual-function unit 502 when switched over) is operating in the separation medium regeneration function, its operating parameters and resulting output are essentially the same as those of the separation medium regeneration unit 416 described above.

Simultaneous operation of the first dual-function unit 502 in the hydrogen bromide separation function and operation of the second dual-function unit 504 in the separation medium regeneration function is effected by cooperatively controlling the position of the in-line valves 512, 514, 522, 524. In particular, valves 512 and 524 are open to allow flow through lines 506 and 518, respectively, while valves 514 and 522 are closed to prevent flow through lines 508 and 516, respectively, when the first dual-function unit 502 is operating in the hydrogen bromide separation function and the second dual-function unit 504 is operating in the separation medium regeneration function.

Simultaneous operation of the second dual-function unit 504 in the hydrogen bromide separation function and operation of the first dual-function unit 502 in the separation medium regeneration function is similarly effected by cooperatively controlling the position of the in-line valves 512, 514, 522, 524. In particular, valves 512 and 524 are closed to prevent flow through lines 506 and 518, respectively, while valves 514 and 522 are open to allow flow through lines 508 and 516, respectively, when the second dual-function unit 504 is operating in the hydrogen bromide separation function and the first dual-function unit 502 is operating in the separation medium regeneration function.

The system operator switches over (i.e., swings) the function of the two units 502, 504 by operation of the valves 512, 514, 522, 524 in the above-described manner. The function switch-over is performed at a point in time preferably before the separation medium reaches its hydrogen bromide-rich gas loading limit (i.e., saturation) and/or before the separation medium exhibits a substantially diminished ability to adsorb additional hydrogen bromide-rich gas.

When the first dual-function unit 502 is operating in the hydrogen bromide separation function and the second dual-function unit 504 is operating in the separation medium regeneration function, the system operator may monitor the first residual gas outlet line 510 and/or the second hydrogen bromide-rich gas outlet line 518 to determine the function switch-over point. However, function switch-over is preferably performed on a timed basis, preferably selecting a switch-over time before the hydrogen bromide concentration in the residual gas of the first residual gas outlet line 510 exceeds about 0 to 1 mol %, or the hydrogen bromide concentration in the hydrogen bromide-rich gas of the second hydrogen bromide-rich gas outlet line 518 falls below about 90 to 100%.

Similarly, when the second dual-function unit 504 is operating in the hydrogen bromide separation function and the first dual-function unit 502 is operating in the separation medium regeneration function, the system operator may monitor the second residual gas outlet line 520 and/or the second hydrogen bromide-rich gas outlet line 508 to determine the function switch-over point. However, function switch-over is preferably performed on a timed basis, preferably selecting a switch-over time before the hydrogen bromide concentration in the residual gas of the second residual gas outlet line 520 exceeds about 0 to 1%, or the hydrogen bromide concentration in the hydrogen bromide-rich gas of the first hydrogen bromide-rich gas outlet line 508 falls below about 90 to 100%

A full cycle of the system 500, and correspondingly the hydrogen bromide recovery stage, operating in the swing regeneration mode is completed when the function switch-over has occurred twice, i.e., when each unit 502, 504 has completed one full term of the hydrogen bromide separation function and one full term of the separation medium regeneration function. The system 500 repeats additional cycles in a continuous manner as long as the system 500 remains in operation. As such, the swing regeneration mode of operation mimics a continuous operating mode of operation.

The hydrogen bromide recovery stage is preferably operated in the swing regeneration mode in a manner which sets the cycle time and operating conditions of the units when performing the hydrogen bromide separation function at values sufficient to enable substantial loading of the hydrogen bromide-rich gas on the separation medium, but without significant break-through of hydrogen bromide in the residual gas. The cycle time and operating conditions of the units when performing the separation medium regeneration function are likewise set at values sufficient to enable substantial desorption of the hydrogen bromide-rich gas from the separation medium within the units. This results in optimal utilization of the separation medium.

The system 500 is shown in FIG. 3 and described above for purposes of illustration as having two dual-function units. However, it is understood that the present invention is not limited in this manner. Although not shown, it is within the purview of a skilled artisan and within the scope of the present invention to employ an interconnected network of three or more dual-function units to increase the capacity and/or efficiency of the hydrogen bromide recovery stage. All the units would preferably continue to operate in parallel in cooperation with one another. However, the individual operation of each unit is substantially the same as described above with respect to the two units and achieves substantially the same result as is apparent to a skilled artisan.

The use of three or more dual-function units has the added advantage of enabling staggered operation which, in particular, advantageously enables depressurization and repressurization of each unit between the adsorption and desorption steps. Staggered operation also advantageously enables the operator to purge residual gas remaining within the unit after each adsorption or desorption step to minimize the loss of residual gas from the system 500 and to avoid interruption of flow between during the purging and/or depressurization step as will be evident to a skilled artisan.

In another alternate embodiment of the hydrogen bromide recovery stage shown conceptually in FIG. 1, the hydrogen bromide-rich gas is separated and recovered from the feed gas by cryogenic means. Although this embodiment is not shown in the drawings, practice of this embodiment is effected simply by replacing the hydrogen bromide separation and separation medium regeneration units 414, 416 of the system 410 or the first and second dual-function units 502, 504 of system 500 with a conventional cryogenic fractionation unit. The feed gas is introduced directly into the cryogenic fractionation unit via a feed gas line which is identical to the feed gas line 412 of system 410 or 500. The resulting hydrogen bromide-rich gas is discharged from the cryogenic fractionation unit and conveyed to the thermal oxidation stage shown conceptually in FIG. 1 via a hydrogen bromide-rich gas line which is identical to the hydrogen bromide-rich gas line 434 of system 410 or 500. The cryogenic fractionation unit is typically operated at a pressure in the range of about 40 bar to about 5 bar and a minimum temperature in a range of about −50° C. to about −150° C.

Regardless of which above-recited embodiment of the hydrogen bromide recovery stage is employed in the hydrogen bromide conversion system used to practice the method of the present invention, the initial hydrogen bromide-rich gas resulting from the hydrogen bromide recovery stage is fed to the thermal oxidation stage via the hydrogen bromide-rich gas line 434 shown in FIG. 2 or 3.

In some cases the feed gas provided at the feed gas line 412 may have a sufficiently high hydrogen bromide concentration that the hydrogen bromide recovery stage is unnecessary. In these cases, the feed gas line 412 bypasses the hydrogen recovery stage and introduces the feed gas directly into the hydrogen bromide-rich gas line 434 for feeding to the thermal oxidation stage. As such, the initial hydrogen bromide-rich gas and the feed gas, i.e., hydrogen bromide-containing gas, are the same in these cases.

Referring to either FIG. 2 or 3, the thermal oxidation stage of both embodiments includes a thermal oxidation unit 436 positioned at the downstream end of the hydrogen bromide-rich gas line 434. The thermal oxidation unit 436 is serially partitioned into a mixing zone 438 and a thermal oxidation zone 440. The thermal oxidation unit 436 has an oxygen-containing gas line 442 in fluid communication with the mixing zone 438 which enables introduction of an oxygen-containing gas into the mixing zone 438. The oxygen-containing gas may be substantially any gas containing oxygen including pure oxygen and mixtures of oxygen with other gases, although the oxygen-containing gas is preferably a dry gas. Although the oxygen-containing gas may be pure oxygen as noted above, a preferred oxygen-containing gas is commonly air which is a more cost-effective and operationally convenient alternative.

The thermal oxidation unit 436 optionally comprises a first trim heater 444 and a second trim heater 446, which are alternately termed start-up heaters. The optional first trim heater 444 is positioned in the hydrogen bromide-rich gas line 434 to preheat the hydrogen bromide-rich gas entering the mixing zone 438, while the optional second trim heater 446 is positioned in the oxygen-containing gas line 442 to preheat the oxygen-containing gas entering the mixing zone 438. The thermal oxidation unit 436 also optionally comprises a pilot burner 448 and a liquid spray injector 450 positioned in the thermal oxidation zone 440. A pilot burner fuel line 452 is in communication with the optional pilot burner 448 to supply a conventional fuel to the pilot burner 448.

Operation of the thermal oxidation unit 436 is initiated by introducing the hydrogen bromide-rich gas and the oxygen-containing gas to the mixing zone 438 via the hydrogen bromide-rich gas line 434 and oxygen-containing gas line 442, respectively. Mixing of the hydrogen bromide-rich gas and oxygen-containing gas in the mixing zone 438 is preferably achieved by one of any number of conventional mixing devices which are known to a skilled artisan. Exemplary mixing devices having utility herein include jet-type injectors, swirl-stabilized mixers, venturi-eductors and the like. The resulting gas mixture is termed a thermal oxidation feed gas herein. The thermal oxidation feed gas preferably has a molar ratio of hydrogen bromide to oxygen in a range of about 4:1.05 to 4:1.5 and more preferably in a range of about 4:1.1 to 4:1.2. In other words, the thermal oxidation feed gas preferably has about 5% to 50% excess oxygen, and more preferably about 10% to 20% excess oxygen over the stoichiometric requirement for complete reaction.

If the optional first trim heater 444 is provided in the hydrogen bromide-rich gas line 434, the hydrogen bromide-rich gas is preferably preheated to a temperature in a range of about 150° C. and about 250° C. upstream of the mixing zone 438. In the absence of preheating, the hydrogen bromide-rich gas is preferably introduced into the mixing zone 438 at a temperature in a range of about 70° C. and about 150° C. In either case, the hydrogen bromide-rich gas is preferably introduced into the mixing zone 438 at a pressure in a range of about 1 and about 20 bar.

If the optional second trim heater 446 is provided in the oxygen-containing gas line 442, the oxygen-containing gas is preferably preheated to a temperature in a range of about 150° C. and about 250° C. upstream of the mixing zone 438. In the absence of preheating, the oxygen-containing gas is preferably introduced into the mixing zone 438 at a temperature in a range of about 70° C. and about 150° C. In either case, the oxygen-containing gas is preferably introduced into the mixing zone 438 at a pressure in a range of about 1 and about 20 bar.

Preheating the hydrogen bromide-rich gas and oxygen-containing gas to within the above-recited temperature ranges advantageously enables initiation of the thermal oxidation reaction when the feed mixture is further heated by mixing with recirculated hot reaction gases within the thermal oxidation zone 440 of the thermal oxidation unit 436. Optionally, a small amount of catalyst may be employed in the mixing zone 438 or in the inlet end of thermal oxidation zone 440 to initiate the thermal oxidation reaction within the mixing zone 438 or inlet end of the thermal oxidation zone 440, as further described, below. The thermal oxidation reaction is characterized as a hydrogen bromide oxidation reaction by the following reaction equation:

4HBr(g)+O₂(g)→2Br₂(g)+2H₂O

Upon formation of the thermal oxidation feed gas in the mixing zone 438, the thermal oxidation feed gas is promptly transferred to the thermal oxidation zone 440. The thermal oxidation zone 440 is preferably an enclosed vessel, chamber, container, or the like having a refractory liner suitable for high-temperatures, such as employed in conventional thermal oxidization units for waste gas oxidation processes. Where the mixing zone 438 is simply a mechanical mixing device of the type recited above, the mixing device discharges the thermal oxidation feed gas directly into the thermal oxidation zone 440.

The thermal oxidation feed gas is preferably discharged into the thermal oxidation zone 440 as a jet with sufficient kinetic energy to impart a recirculation of hot reaction gases within the thermal oxidation zone 440. Gas recirculation causes heating and mixing of the thermal oxidation feed gas with the hot recirculation gases, thereby achieving a minimum homogeneous thermal oxidation reaction initiation temperature of about 650° C. to about 800° C. This initiates thermal oxidation reaction and allows it to become self-sustaining within the thermal oxidation zone 440.

A small pilot burner 448 may be optionally operated to preheat the thermal oxidation zone 440 during startup and to subsequently insure initiation of the thermal oxidation reaction. Alternatively, or in addition, a small amount of catalyst may be employed within the mixing zone 438 or at the inlet end of thermal oxidation zone 440 to initiate the reaction. Non-limiting examples of catalysts which may be used to initiate the reaction may include heat-stable transition metal oxides such as iron oxides, nickel oxides, chromium oxides or rare-earth oxides and the like, or platinum, ruthenium or other platinum group metals or combinations thereof.

In any case, it is preferable to use either: 1) preheating of the thermal oxidation feed gas and mixing with recirculation gases; 2) start-up heating of the thermal oxidation feed gas by means of the pilot burner 448; or 3) preheating of the thermal oxidation feed gas in combination with a small amount of initiation catalyst, or any combination thereof, so that the minimum homogeneous thermal oxidation reaction initiation temperature of about 650° C. to about 800° C. is achieved, thereby initiating the homogeneous thermal oxidation reaction within the thermal oxidation zone 440.

Once the thermal oxidation reaction initiation temperature is achieved, the thermal oxidation reaction proceeds fairly rapidly within the thermal oxidation zone 440 if heat losses are minimized. As such, the reaction conditions within the thermal oxidation zone 440 approach adiabatic conditions. Due to the large exothermic heat of reaction, the temperature of the reacting gases rises rapidly above 800° C. and may reach up to a range of about 1000° C. to 1200° C. as the reaction proceeds, depending on the particular composition of the reacting gases and the presence of excess oxygen and inert gases such as nitrogen, which may be present if air is utilized as the oxidizing gas.

In order to achieve substantial conversion of the hydrogen bromide to elemental bromine in accordance with the above-recited hydrogen bromide oxidation reaction equation, it is preferable to maintain the thermal oxidation zone 440 at a temperature in a range of about 950° C. to about 1100° C. and at a pressure in a range of about 1 and about 20 bar. In addition a preferred residence time of the thermal oxidation feed gas in the thermal oxidation zone 440 is in a range between about 1 second and about 10 seconds.

A preferred means for maintaining the relatively high reaction temperature in the thermal oxidation zone 440 is to continuously recirculate a portion of the hot thermal oxidation reaction gases at the downstream end of the thermal oxidation zone 440 back to the upstream end of the thermal oxidation zone 440 as shown by the recirculation arrows in FIGS. 2 and 3. The recirculated hot gases contact and mix with the fresh thermal oxidation feed gas entering the thermal oxidation zone 440 from the mixing zone 438, thereby heating the fresh thermal oxidation feed gas and recirculated gases to the thermal oxidation reaction initiation temperature and allowing the reaction to become self-sustaining.

Internal baffles can optionally be mounted in the thermal oxidation zone 440 to effect recirculation of the hot thermal oxidation reaction gases. The thermal oxidation zone 440 can also optionally be subdivided into a plurality of sub-zones. The downstream sub-zones provide additional residence time within the thermal oxidation zone 440 for the thermal oxidation reaction to occur, if desired. Packing composed of ceramics or other refractory materials suitable for high-temperature use can also be placed in the thermal oxidation zone 440 to promote mixing and heating of the gases therein and provide stability of operation due to thermal inertia. The packing can be either a randomly-dumped packing or a structured packing.

Additional optional heat sources can also be provided to supplement the recirculated hot thermal oxidation reaction gases in maintaining the reaction temperature in the thermal oxidation zone 440. Furthermore, when a small amount of catalyst is employed in the mixing zone 438 or in the inlet end of the thermal oxidation zone 440, as noted above, in conjunction with the optional first and second trim heaters 444, 446, the practitioner can achieve the desirable effect of initiating the thermal oxidation reaction in the mixing zone 438 and of reaching and maintaining the desired reaction temperature in the thermal oxidation zone 440. This can reduce or eliminate the requirement for recirculating hot gases within the thermal oxidation zone 440, if desired, or compensate for minor deviations or disruptions in the flow or composition of the hydrogen bromide-rich gas and/or oxidizing gas.

The optional pilot burner 448 can also be employed as a supplemental heat source in place of, or in addition to, the optional first and second trim heaters 444, 446. A conventional fuel is fed to the pilot burner 448 positioned at the upstream end of the thermal oxidation zone 440 proximal to the mixing zone 438 via the pilot burner fuel line 452. Burning the conventional fuel in the pilot burner 448 enables it to function as a trim heater to maintain a minimum reaction temperature in the thermal oxidation zone 440 in a controlled manner, if desired. In the absence of preheating, the pilot burner 448 can also be utilized to initiate the thermal oxidation reaction as noted above.

Yet another alternate supplemental heat source is waste heat from an associated upstream process. For example, a gaseous alkane conversion process for producing liquid hydrocarbons, such as a bromination/synthesis process, also typically produces waste heat which can have utility in the thermal oxidation zone 440 of the present method.

The thermal oxidation of dry gaseous hydrogen bromide as in the present reaction is characterized by a large temperature increase moving downstream across the thermal oxidation zone 440. This temperature gradient is attributable to the exothermic nature of the reaction and a relatively low heat capacity of the reactants due to the absence or relatively small amount of steam present in the thermal oxidation feed gas. Although this temperature gradient can be used advantageously by recirculating the hot gases as described above to initiate the thermal oxidation reaction in the thermal oxidation zone 440 and/or to maintain the reaction temperature in the thermal oxidation zone 440, the high temperature generated in the thermal oxidation zone 440 also has inherent disadvantages.

The high temperature in the thermal oxidation zone 440 is destructive to known catalysts, such as CuBr₂ or CeBr₃, which are most active for high conversion for the hydrogen bromide conversion reaction. In particular, such catalysts are unstable at the contemplated operating temperature in the thermal oxidation reaction zone 440 which would cause a loss of catalyst activity as well as possible fouling of downstream process equipment. Accordingly, in a preferred embodiment, the thermal oxidation zone 440 is substantially catalyst-free. However, in an alternate embodiment where an optional packing is placed in the thermal oxidation zone 440, a portion or layer of the packing near the inlet end of the thermal oxidation zone 440 can be coated with a catalytically active substance, which exhibits greater stability at the high temperature in the thermal oxidation zone 440 than the above-recited most active catalysts. These catalytically active substances initiate and/or promote the high-temperature thermal oxidation reaction in the thermal oxidation zone 440. Exemplary classes of catalytically active substances having utility in the present embodiment include transition metal oxides or rare-earth oxides, which do not form volatile metal bromides or volatile rare-earth bromides. Alternatively, a catalytic metallic gauze, mesh, or the like formed from the same catalytically active substance as a platinum group metal can be placed in the thermal oxidation zone 440 to initiate and/or promote the high-temperature thermal oxidation reaction.

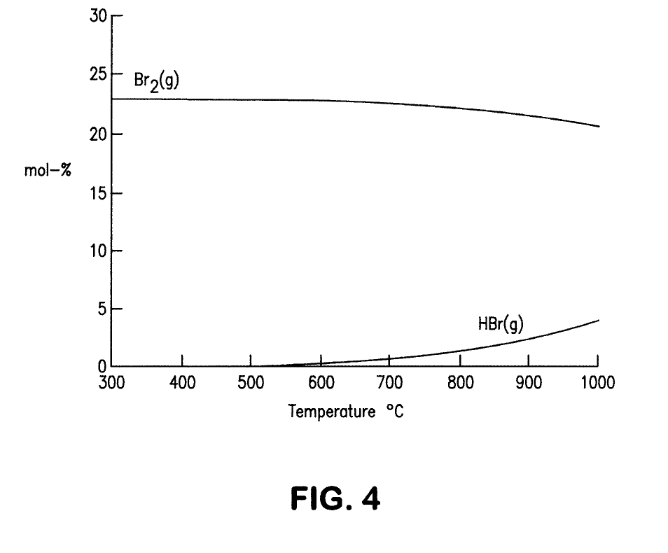

Another inherent disadvantage of the high temperature generated in the thermal oxidation zone 440 is the equilibrium limitations imposed on the hydrogen bromide conversion reaction at the high operating temperature contemplated in the thermal oxidation reaction zone 440. FIG. 4 graphically displays the results of a thermodynamic equilibrium calculation versus temperature for the thermal oxidation reaction of hydrogen bromide with air 20% in excess of the stoichiometric amount that would be required for complete reaction of the hydrogen bromide. It is apparent that the extent of the reaction is limited to less than 100% completion at temperatures above about 500° C. Thus, for example, this temperature equilibrium limitation would limit the hydrogen bromide conversion rate to a theoretical maximum of about 91%, depending on gas-kinetic reaction rates, at an adiabatic thermal oxidation reaction temperature of about 1000° C.

This equilibrium limitation can be mitigated to some extent by employing the optional liquid spray injector 450 in the thermal oxidation zone 440 as shown in FIGS. 2 and 3. In accordance with an alternate embodiment of the present process, a spray of liquid droplets are introduced into the liquid spray injector 450 via the thermal oxidation zone 440. The sprayed liquid droplets evaporate and partially cool the thermal oxidation reaction gases. This partial cooling may have the desired effect of moderating the temperature rise within the thermal oxidation zone 440, thereby reducing the equilibrium limitation illustrated in FIG. 4 and described above. However, the lower temperature likely reduces the kinetic rate of reaction, thereby requiring additional residence time to achieve a desired higher conversion.

In accordance with one embodiment, the liquid sprayed into the thermal oxidation zone 440 is an aqueous hydrobromic acid or some other bromide-containing liquid waste stream. The aqueous hydrobromic acid can be obtained from a hydrogen bromide recovery process. An exemplary hydrogen bromide recovery process is the removal of residual hydrogen bromide from a liquid hydrocarbon product stream by water washing the stream in a multi-stage countercurrent extraction column. In any case, the vaporized bromide-containing compounds in the liquid spray are thermally oxidized in the thermal oxidation zone 440 along with the hydrogen bromide in the hydrogen bromide-rich gas to supplement the amount of elemental bromine produced in the thermal oxidation zone 440.

The mixing zone 438 and thermal oxidation zone 440 are shown in FIGS. 2 and 3 and described above for purposes of illustration as being integrated into a single unitary structure. However, it is understood that the present invention is not limited in this manner. Although not shown, it is within the purview of a skilled artisan and within the scope of the present invention to house the mixing zone 438 and thermal oxidation zone 440 in separate structures in fluid communication with one another.

The hot thermal oxidation reaction gases at the downstream end of the thermal oxidation zone 440 are termed the thermal oxidation effluent gas. The thermal oxidation effluent gas comprises elemental bromine, steam, unconverted hydrogen bromide and excess oxygen. When air is the oxygen-containing gas, the thermal oxidation effluent gas further comprises other air constituents in addition to oxygen, such as nitrogen and carbon dioxide. The thermal oxidation effluent gas is preferably at a temperature in a range of about 950° C. to about 1100° C. and at a pressure in a range of about 1 and about 100 bar. The amount of elemental bromine in the thermal oxidation gas preferably corresponds to a hydrogen bromide conversion rate in the thermal oxidation stage in a range of about 80% to about 95% of the hydrogen bromide which is present in the hydrogen bromide-rich gas discharged into the hydrogen bromide-rich gas line 434 from the hydrogen bromide recovery stage.

The thermal oxidation unit 436 additionally has a thermal oxidation effluent gas outlet port 454. A thermal oxidation effluent gas line 456 extends from the thermal oxidation effluent gas outlet port 454 to the catalytic oxidation stage shown conceptually in FIG. 1. The catalytic oxidation stage of the present embodiments includes a catalytic reactor 458 positioned at the downstream end of the thermal oxidation effluent gas line 456 which has a thermal oxidation effluent gas inlet port 460. As such, the thermal oxidation effluent gas is conveyed from the thermal oxidation unit 436 to the catalytic reactor 458 via the thermal oxidation effluent gas outlet port 454 and the thermal oxidation effluent gas line 456 and is introduced into the catalytic reactor 458 via the thermal oxidation effluent gas inlet port 460.

A waste-heat recovery heat exchanger 462 is positioned in the thermal oxidation effluent gas line 456 which cools the thermal oxidation effluent gas to a temperature of about 250° C. to 335° C., thereby recovering waste heat from the thermal oxidation zone 440. The temperature of the heat exchange surface in the waste-heat recovery heat exchanger 462 is preferably maintained above the dew-point of the thermal oxidation effluent gas so that less expensive materials can be used in the construction of the waste-heat recovery heat exchanger 462, such as nickel or nickel-containing metal alloys.

The catalytic reactor 458 is an enclosed vessel, chamber, container, or the like containing a bed of a highly active oxidation catalyst. Representative classes of highly reactive oxidation catalysts having utility in the catalytic reactor include transition metal oxides, transition metal bromides, rare-earth oxides, rare-earth bromides or combinations thereof, further these may be used directly or dispersed on an oxide, carbide or nitride support. Among these representative highly reactive oxidation catalysts, CuO/CuBr₂ supported on alumina or CeBr₃ supported on alumina or zirconia are preferred. The thermal oxidation effluent gas adiabatically contacts the highly active oxidation catalyst bed in the catalytic reactor 458 at a reactor inlet temperature in a range of about 250° C. to about 345° C. and a pressure in a range of about 1 bar to about 20 bar to essentially complete the conversion of the remaining hydrogen bromide in the thermal oxidation effluent gas. As such, the highly active oxidation catalyst completes the last about 5% to about 20% of the hydrogen bromide conversion reaction in the above-recited reaction equation.

Since the amount of unconverted hydrogen bromide contacting the catalyst bed in the catalytic reactor 458 is a relatively small fraction of the total hydrogen bromide initially present in the feed gas, the temperature rise across the uncooled adiabatic catalytic reactor 458 is relatively small. Therefore, the outlet temperature of the catalytic reactor 458 can preferably be maintained in a range of about 300° C. to 450° C. and more preferably in a range of about 325° C. to 350° C., which maintains the long-term stability of the oxidation catalyst.

The catalytic reactor 458 has a catalytic reactor effluent gas outlet port 464 through which the catalytic reactor effluent gas is discharged. Since the thermal oxidation and catalytic oxidation stages achieve essentially complete conversion of hydrogen bromide in the feed gas to elemental bromine, the catalytic reactor effluent gas is comprised primarily of elemental bromine, steam, excess oxygen, and any remaining unreactive constituents such as air constituents other than oxygen. A catalytic reactor effluent gas line 466 extends from the catalytic reactor effluent gas outlet port 464 to the separation and product recovery stage shown conceptually in FIG. 1.

The separation and product recovery stage of the present embodiment comprises a quench/condenser 468, a three-phase gas/liquid/liquid separator 470, a gas treatment unit 472 and an aqueous liquid treatment unit 474. The catalytic reactor effluent gas is conveyed to the quench/condenser 468 via the catalytic reactor effluent gas outlet port 464 and the catalytic reactor effluent gas line 466. The catalytic reactor effluent gas is quenched in the quench/condenser 468 and cooled to a temperature in a range of about 5° C. to about 60° C. at a pressure in a range of about 1 to about 20 bar, thereby condensing a substantial portion of the gas into a liquid. The result is a three-phase mixture comprising a gas phase, a heavier elemental bromine liquid phase, and a lighter aqueous liquid phase. The three-phase mixture is conveyed to the three-phase gas/liquid/liquid separator 470 via a condenser outlet line 476 where the gas phase, aqueous liquid phase and elemental bromine liquid phase are all separated from one another.

The elemental bromine liquid phase is drawn off the bottom of the three-phase gas/liquid/liquid separator 470. The elemental bromine liquid phase is essentially pure elemental bromine in a liquid state containing only a trace of residual dissolved water, typically about 0.3 wt %, and possible traces of unreacted hydrogen bromide. The elemental bromine liquid phase contains the bulk of the elemental bromine product recovered from the above-described upstream process. The elemental bromine product is recovered from the three-phase gas/liquid/liquid separator 470 via an elemental bromine product recovery line 477.

The gas phase, which is drawn off the top of the three-phase gas/liquid/liquid separator 470, comprises primarily oxygen-depleted air, if the oxygen-containing gas in the oxygen-containing gas line 442 is air, or primarily oxygen, if the oxygen-containing gas in the oxygen-containing gas line 442 is pure oxygen. The gas phase also includes any residual elemental bromine which is not condensed in the quench/condenser 468. The concentration of elemental bromine in the gas phase is preferably in a range of about 1 mol % to about 10 mol %.

Higher system operating pressures and lower quench/condensing temperatures will maximize condensation, thereby minimizing the residual bromine concentration in the gas phase leaving the three-phase separator. Conversely, operation at lower pressures and higher quench/condensing temperatures will result in higher residual bromine vapor concentration in the gas-phase.

The gas phase is conveyed to the gas treatment unit 472 via a separator gas phase outlet line 478. The gas treatment unit 472 is substantially any conventional operational unit capable of near complete recovery of a halogen from a gas stream, such as an absorption gas scrubbing unit, a solid bed absorption unit, or the like, selected by a skilled practitioner for the specified size and operating conditions of the system 410. The gas treatment unit 472 removes the elemental bromine from the gas phase. The remaining substantially bromine-free gas phase is discharged from the gas treatment unit 472 via a vent line 480, while the residual elemental bromine is recovered from the gas treatment unit 472 and fed into the elemental bromine product recovery line 477 via a gas treatment bromine recovery line 482.

The lighter aqueous liquid phase, which is decanted from the heavier elemental bromine liquid phase in the three-phase gas/liquid/liquid separator 470, is comprised primarily of water and some residual elemental bromine dissolved therein. A first portion of the aqueous liquid phase is returned by an in-line pump 484 to the quench/condenser 468 via a condenser recycle line 492 after first cooling the recycled first portion of the aqueous liquid phase in an in-line cooler 494. The cooled first portion of the aqueous liquid phase functions as a cooling medium for the quench/condenser 468. A second portion of the lighter aqueous liquid phase is conveyed by the in-line pump 484 to the aqueous liquid treatment unit 474 via a separator aqueous liquid phase outlet line 486. The aqueous liquid treatment unit 474 is substantially any conventional operational unit capable of recovering a dissolved halogen from a liquid stream such as a distillation column, a reboiled stripper column, a solid bed absorption unit, or the like, selected by a skilled practitioner for the specified size and operating conditions of the system 410.

The aqueous liquid treatment unit 474 removes the dissolved elemental bromine from the aqueous liquid phase and the elemental bromine is recovered from the aqueous liquid treatment unit 474 via an aqueous liquid treatment bromine recovery line 488 which feeds into the elemental bromine product recovery line 477. The substantially bromine-free aqueous liquid phase is discharged from the aqueous liquid treatment unit 474 via a drain line 490.

Alternatively, a portion of the substantially bromine-free aqueous liquid phase may be returned to the quench/condenser 468 via the condenser recycle line 492 after first cooling the recycled substantially bromine-free aqueous liquid phase in the in-line cooler 494. This may be done in lieu of using the aqueous liquid phase leaving the three-phase separator 470 which contains dissolved bromine. In this alternate embodiment (not shown) less expensive materials could be employed in the construction of the in-line cooler 494 due to the removal of corrosive dissolved bromine from the aqueous liquid phase. However this advantage is offset by the increased expense necessitated by a larger capacity aqueous liquid treatment unit 474.

The following examples demonstrate the scope and utility of the present invention which enable the conversion of hydrogen bromide to elemental bromine. However, these examples are not to be construed as limiting the scope of the present invention.

### Example 1

A quartz tube having a 1 inch ID (21 mm) and is 46 inches in length (1168 mm) is employed as a reactor in the present example. The first half of the tube length is a packed section containing a packing of ¼-inch (6.4 mm) ceramic Berl saddles. The remaining half of the tube length is open. The tube is placed in an electric furnace having two heated zones. Each heated zone is 12 inches (305 mm) in length. The packed section of the tube is positioned within the first heated zone and has a void volume of approximately 55 cm³. The open section of the tube is positioned within the second heated zone and has a volume of approximately 105 cm³.

A series of thermal test runs, T-1 through T-7, are performed in the reactor, each test run having a different set of operating parameters. In each thermal test run, excess air and gaseous hydrogen bromide are fed to the packed section of the tube at a desired feed rate. An initial length of the packed section functions as a reactant mixing and preheat zone. A 0.25-inch (6.4 mm) OD internal quartz thermowell mounted in the packed section and a sliding thermocouple are cooperatively employed to determine the temperature profile of the packed section. It is observed that the feed gases closely approach the furnace temperature when the feed gases reach a point about 4 inches (102 mm) into the packed section within the heated zone. Thus, the initial 4 inches of the packed section define a reactant mixing and preheat zone, while the final 8 inches (203 mm) of the packed section, which have a 50% void volume, and the entire length of the open section define a thermal oxidation reactor zone. As such, the quartz tube functions in the manner of the thermal oxidation unit 436 serially partitioned into the mixing zone 438 and thermal oxidation zone 440 as shown in FIGS. 2 and 3.

The residence time of the gaseous reactants in the reactor zone is calculated. The resulting gaseous effluent discharged from the reactor zone is routed to a water-cooled condenser where a liquid elemental bromine phase and an aqueous liquid phase are obtained as the condenser effluent. The condenser effluent is routed through a series of five gas-liquid impingers to recover the elemental bromine and any unreacted hydrogen bromide from the condenser effluent. The first two impingers contain 0.1 N H₂SO₄, the third impinger contains water, and the fourth and fifth impingers contain an aqueous mixture of 5% NaOH and 5% Na₂SO₃.

The liquid elemental bromine phase and the aqueous liquid phase are weighed and analyzed. This data along with analyses of the five impinger solutions are used to determine the total amount of elemental bromine and hydrogen bromide recovered from the thermal oxidation reactor zone and, hence, the percentage of hydrogen bromide conversion in the thermal oxidation reactor zone.

The operating parameters and results of each test run are set forth below in Table 1.

TABLE 1

| | Reactor | HBr | Excess | Res. | HBr | Air |
|---|---|---|---|---|---|---|
| | Temp. | Conv. | Air | Time | Feed | Feed |
| Run | ° C. | % | % | sec | cm³/min | cm³/min |
| T-1 | 950 | 82 | 21 | 3.4 | 292 | 422 |
| T-2 | 995 | 85 | 18 | 3.3 | 294 | 412 |
| T-3 | 1084 | 84 | 19 | 3.1 | 292 | 412 |
| T-4 | 900 | 65 | 19 | 3.6 | 292 | 412 |
| T-5 | 849 | 51 | 18 | 3.8 | 294 | 412 |
| T-6 | 898 | 65 | 17 | 3.6 | 296 | 412 |
| T-7 | 997 | 82 | 18 | 3.3 | 294 | 412 |

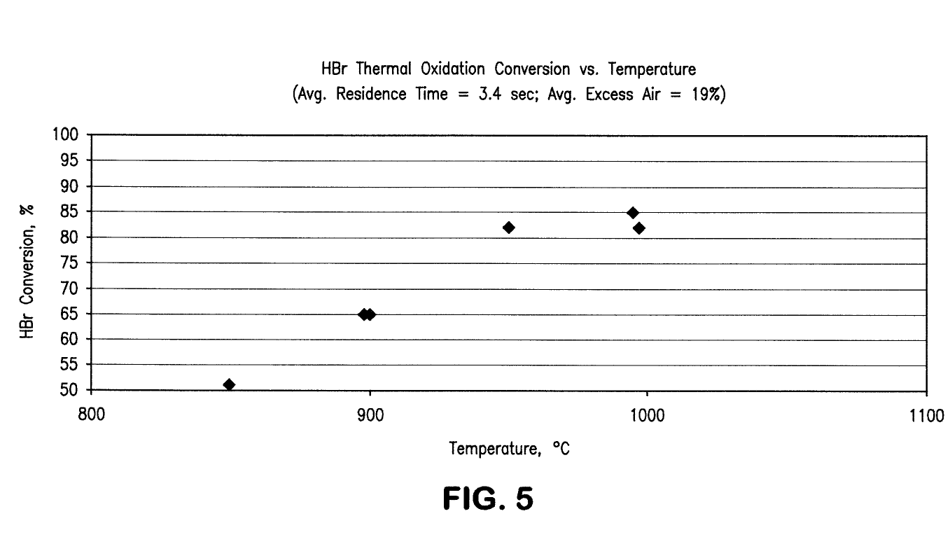

FIG. 5 is a graphical depiction showing the effect of reactor temperature on hydrogen bromide conversion in the thermal oxidation reactor zone.

### Example 2

A series of additional thermal test runs, T-7 through T-10, are performed in the same apparatus and using the same analytical methods as Example 1. However, the focus of the present example is to determine the effect that varying the amount of excess air over stoichiometric has on hydrogen bromide conversion in the thermal oxidation reactor zone. In addition, the feed gas throughput in test run T-10 is reduced to determine the effect of increased reactant residence time in the thermal oxidation reactor zone at high excess air on hydrogen bromide conversion.

The operating parameters and results of the additional test runs are set forth below in Table 2. The results of test runs T-2 and T-7 are also repeated from Table 1 for comparison.

TABLE 2

| | Reactor | HBr | Excess | Res. | HBr | Air |
|---|---|---|---|---|---|---|
| | Temp. | Conv. | Air | Time | Feed | Feed |
| Run | ° C. | % | % | sec | cm³/min | cm³/min |
| T-2 | 995 | 85 | 18 | 3.3 | 294 | 412 |
| T-7 | 997 | 82 | 18 | 3.3 | 294 | 412 |
| T-8 | 996 | 79 | 12 | 3.3 | 309 | 412 |
| T-9 | 998 | 90 | 44 | 3.0 | 285 | 489 |
| T-10 | 998 | 92 | 49 | 4.8 | 178 | 316 |

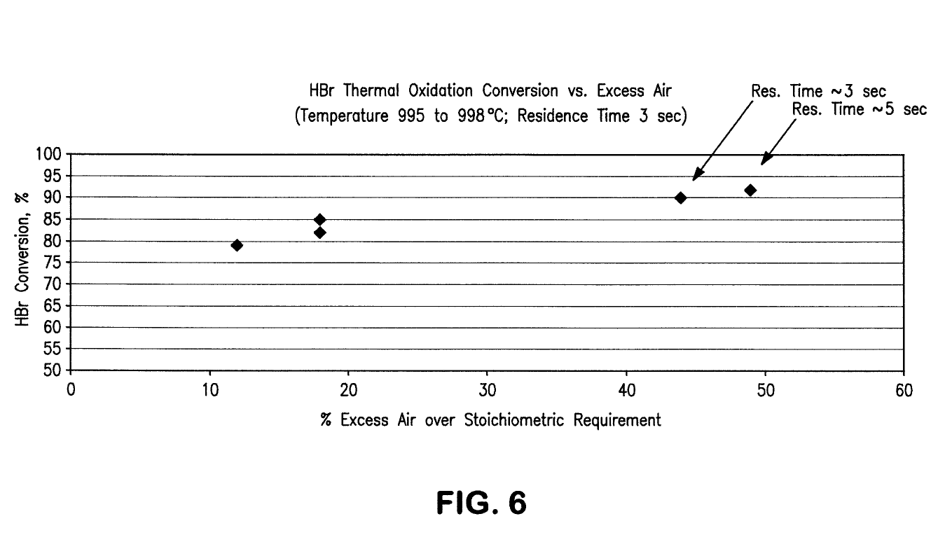

FIG. 6 is a graphical depiction showing the effect of the amount of excess air on hydrogen bromide conversion in the thermal oxidation reactor zone.

### Example 3

The packing of the quartz tube of Examples 1 and 2 is modified by filling the entire first heated zone of the tube with ceramic Berl saddles having the same characteristics as Examples 1 and 2. The first 5 inches (127 mm) of the second heated zone of the tube is packed with 50 cm³ of catalyst and the remaining 7 inches (178 mm) of the second heated zone are filled with the ceramic Berl saddles. As such, the quartz tube simulates the function of the catalytic reactor 458 as shown in FIGS. 2 and 3.

A series of catalytic test runs, C-1 through C-4, are performed in the apparatus using the same analytical methods as above to determine the effect that catalysts, operating within a lower range of temperatures, have on hydrogen bromide oxidation. The operating parameters and results of the catalytic test runs are set forth below in Table 3.

TABLE 3

| | | Reactor | HBr | Excess | GHSV | HBr Feed | Air Feed |
|---|---|---|---|---|---|---|---|
| | | Temp. | Conv. | Air | at STP | at STP | at STP |
| Run | Catalyst | ° C. | % | % | hr⁻¹ | cm³/min | cm³/min |
| C-1 | NiO/alumina | 550 | 98.5 | 31 | 1050 | 300 | 770 |
| C-2 | NiO/alumina | 650 | 97.7 | 31 | 1050 | 300 | 770 |
| C-3 | NiO/alumina | 750 | 94.7 | 31 | 1050 | 300 | 770 |
| C-4 | CuO/alumina | 350 | 98.8 | 33 | 1050 | 296 | 766 |

The results shown in Table 3, indicate that the preferred more active, but less heat-stable, CuO catalyst achieves fairly high conversion at the specified test conditions at a relatively lower temperature of about 350° C. By comparison, the generally less active, but more heat-stable, NiO catalyst must operate at a significantly higher temperature of about 550° C. to achieve similar hydrogen bromide conversion. Further, operating the less active NiO catalyst at higher temperatures between 650° C. and 750° C. or more results in lower, rather than higher hydrogen bromide conversions. This is contrary to what one would expect based on the thermodynamic equilibrium temperature limitation of the hydrogen bromide oxidation reaction illustrated in FIG. 4.

### Example 4

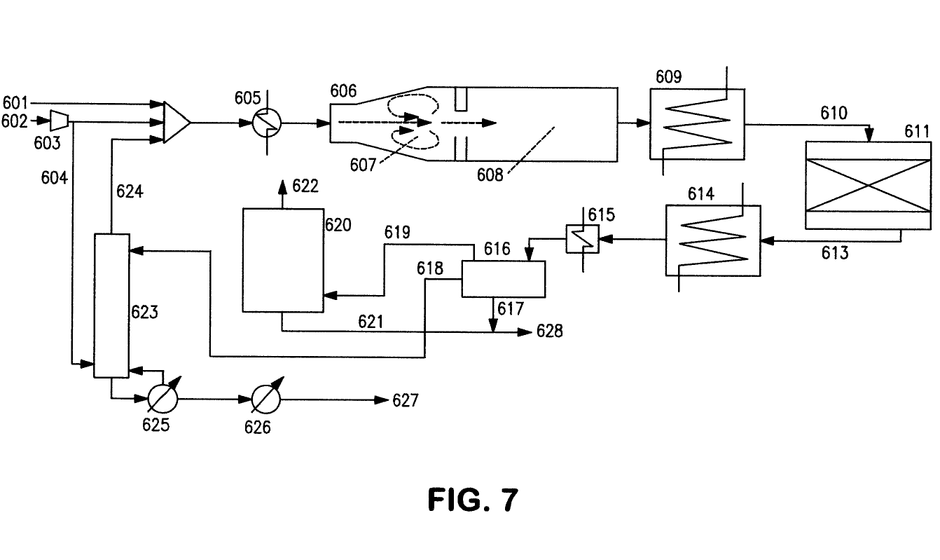

With reference to FIG. 7, Example 4 is an embodiment of the hydrogen bromide conversion method of the present invention shown schematically in a process flow diagram. A computer simulation is used to define the streams referenced in the flow diagram in association with the unit operations of the process as follows:
601 HBr feed: rate=8.65 kg-moles/h; P=5 bar; T=35° C.
602 air feed: rate=11.57 kg-moles/h (20% excess); P=1 bar, T=30° C.
603 air compressor: P_{OUT}=5 bar
604 air to Br₂ stripper: rate=2.34 kg-mole/h; P=5 bar; T=158° C.
605 pre-heater exchanger: T_{OUT}=166° C.
606 thermal oxidation reactor (refractory-lined, etc.)
607 mixer/hot gas recirculation zone: T_{OUT}=732° C.
608 thermal oxidation zone: T_{OUT}=1001° C.
609 high-grade heat recovery exchanger, T_{OUT}=254° C.
610 thermal oxidation reactor effluent: HBr outlet rate=0.866 kg-moles/h (90% conversion to Br₂)
611 catalytic oxidation reactor: T_{OUT}=350° C., P_{OUT}=3.3 bar
612 catalyst bed (CuO, CeBr₃, Cr₂O₃, etc.)
613 catalytic oxidation reactor effluent: HBr outlet rate=0.0029 kg-moles/h (99.97% conversion to Br₂)
614 low-grade heat recovery exchanger: T_{OUT}=115° C.
615 condenser: T_{OUT}=20° C.
616 three-phase separator
617 three-phase separator effluent: liquid Br₂ outlet rate=3.361 kg-moles/hr
618 three-phase separator effluent: aqueous phase outlet rate=4.221 kg-moles/hr (˜0.3 mole % Br₂)
619 three-phase separator effluent: vapor phase outlet rate=10.65 kg-moles/hr (9 mol % Br₂, 4 mol % O₂, 85 mol % N₂, ˜1 mol % H₂O)
620 Br₂ recovery system (circulating, regenerated solvent, cyclically-regenerated solid adsorbent, etc.)
621 Br₂ recovery system effluent: Br₂ outlet rate=0.96 kg-moles/hr (from regenerated solvent, adsorbent, etc.)
622 Br₂ recovery system effluent: scrubbed vent stream outlet rate=9.689 kg-moles/hr (93.2 mol % N₂, 4.5 mol % O₂, ˜1 mol % H₂O, trace Argon, CO₂)
623 aqueous bromine stripping column
624 aqueous bromine stripping column effluent: air-stripped Br₂ vapor outlet rate=2.36 kg-moles/hr (77.5 mol % N₂, 20.4 mol % O₂, ˜0.5% H₂O, ˜0.5 mol % Br₂, trace Argon, CO₂)
625 bromine-stripping column reboiler: T_{OUT}=60° C.
626 stripped water cooler: T_{OUT}=30-50° C.
627 stripped water cooler effluent: stripped water outlet rate=4.198 kg-moles/hr (99.93 mol % H₂O, ˜0.0697 mol % HBr)
628 final product: Br₂ outlet rate=4.323 kg-moles/hr

A feed gas having utility in the above-described embodiments of the present invention has been generally characterized as substantially any gas which contains hydrogen bromide, i.e., a hydrogen bromide-containing gas. As such, the feed gas can be an essentially pure hydrogen bromide gas or a gas mixture containing hydrogen bromide and one or more other constituents. The feed gas is often derived from an upstream process which is either an associated source or an unrelated source. The term “derived”, as used herein with respect to the certain process gas streams, encompasses feeding the hydrogen bromide-containing gas from the upstream process into the feed gas line 412 for processing in the hydrogen bromide recovery stage of the system 410 or 500 where the hydrogen bromide-containing gas has a lower hydrogen bromide concentration than the initial hydrogen bromide-rich gas. The term “derived” also encompasses feeding the hydrogen bromide-containing gas directly from the upstream process into the hydrogen bromide-rich gas line 434 of the system 410 or 500 with little or no additional processing in the case where the initial hydrogen bromide-rich gas and the hydrogen bromide-containing gas are the same.

An upstream process is termed an “associated source” when the hydrogen bromide conversion method of the present invention reconveys its elemental bromine product back to the upstream process from which the feed gas is derived. An upstream process is termed an “unrelated source” when the present hydrogen bromide conversion method conveys its elemental bromine product to some user or destination other than the upstream process from which the feed gas is derived.

A generalized example of an associated upstream source, from which the feed gas can be derived, is a process for converting an organic or inorganic feedstock into more desirable end products. In one such typical conversion process, gaseous alkanes are brominated and the resulting alkyl bromides are catalytically synthesized to form liquid hydrocarbon products. The synthesis reaction produces a byproduct gas generally comprising a mixture of hydrogen bromide and lower molecular weight hydrocarbons which is particularly suitable as a feed gas for the present hydrogen bromide conversion method.

In some cases, as noted above, this byproduct gas is in a suitable condition to be introduced directly into the feed gas line 412 of the system 410 or 500 as the feed gas without substantially any further upstream processing. In other cases it may be desirable to further process the byproduct gas before introducing it into the feed gas line 412 of the system 410 or 500 by performing one or more additional pretreatment steps upstream of the system 410 or 500. For example, such additional pretreatment steps may include heating, cooling, expanding, compressing, concentrating, diluting, drying, introducing additives, or the like. The appropriate selection of such additional pretreatment steps, if any, and the manner of performing them are within the purview of a skilled artisan and are within the scope of the present invention.

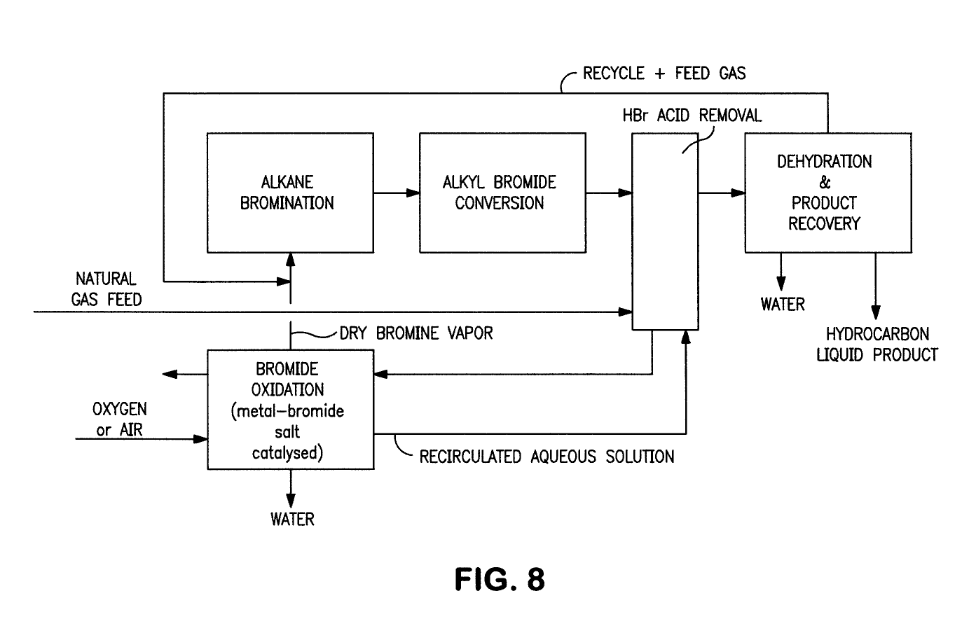

Details of a typical upstream process for producing desirable liquid hydrocarbon products, which can be an associated source of the feed gas for the present hydrogen bromide conversion method, are embodied in U.S. patent application Ser. No. 12/123,924 (Patent Application Publication No. US 2008/0275284 A1) which is incorporated herein by reference. These details are likewise set forth in the description below with reference to FIGS. 8-21.

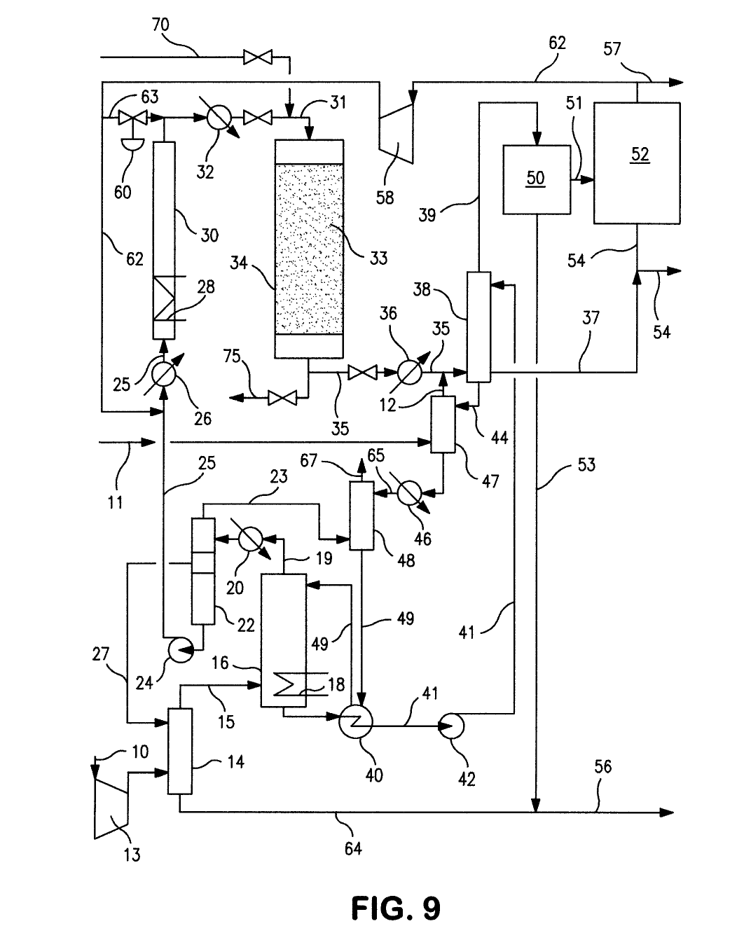

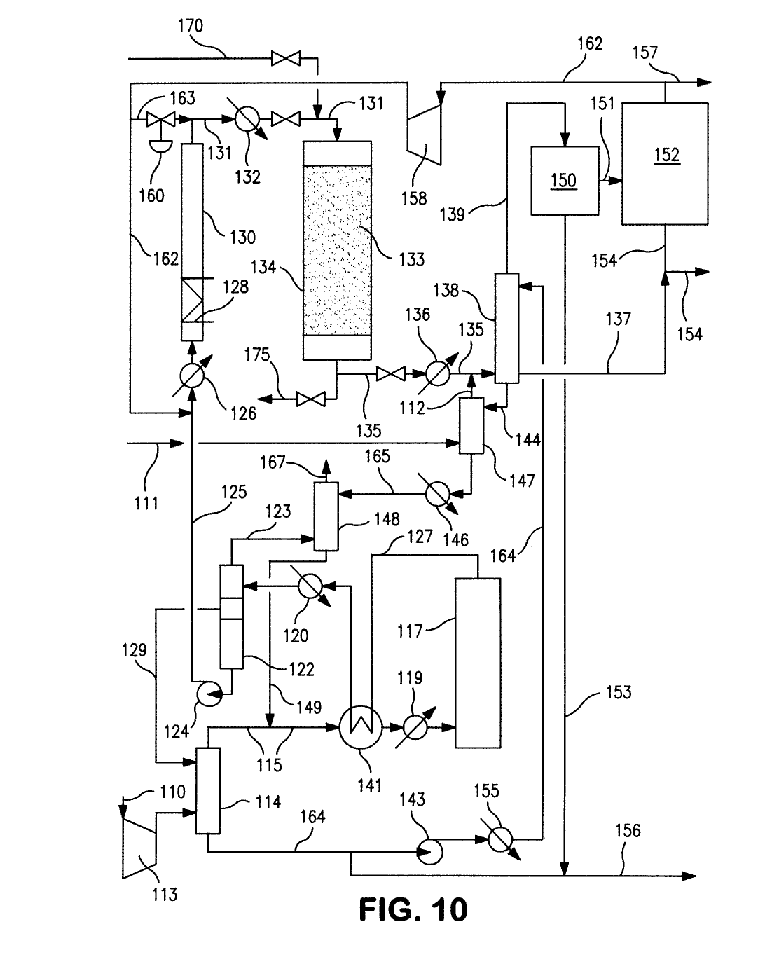

As utilized throughout the following description, the term “lower molecular weight alkanes” refers to methane, ethane, propane, butane, pentane or mixtures thereof. As also utilized throughout this description, “alkyl bromides” refers to mono, di, and tri brominated alkanes. In addition, the feed gas in lines 11 and 111 of the process illustrated in FIGS. 9 and 10, respectively, is preferably natural gas. The natural gas may be treated to remove sulfur compounds and carbon dioxide, although small amounts of carbon dioxide, e.g. less than about 2 mol %, can be tolerated in the feed gas.

A block flow diagram generally depicting an exemplary associated upstream process for producing desirable liquid hydrocarbon products is illustrated in FIG. 8, while specific embodiments of the process are illustrated in FIGS. 9 and 10. Referring to FIG. 8, a gas stream made up of a recycle gas and a feed gas is combined with dry bromine vapor and fed to an alkane bromination reactor. The gas stream preferably comprises lower molecular weight hydrocarbons. The gas stream and dry bromine vapor are reacted in the alkane bromination reactor to produce gaseous alkyl bromides and hydrobromic acid vapors. The resulting gaseous alkyl bromides and hydrobromic acid vapors are fed to an alkyl bromide conversion reactor where the gaseous alkyl bromides are reacted to form higher molecular weight hydrocarbons and additional hydrobromic acid vapors.

The hydrobromic acid vapors are removed from the higher molecular weight hydrocarbons in a hydrobromic acid removal unit by a recirculated aqueous solution. The recirculated aqueous solution carries the hydrobromic acid (or a metal bromide salt if the acid is neutralized by the aqueous solution) to a bromide oxidation unit. If not already neutralized upstream, the hydrobromic acid is neutralized in the bromide oxidation unit to form a metal bromide salt. In any case, oxygen or air is supplied to the bromide oxidation unit to oxidize the metal bromide salt and form elemental bromine, which is recycled to the alkane bromination reactor.

A natural gas feed is also introduced into the hydrobromic acid removal unit with the higher molecular weight hydrocarbons and hydrobromic acid vapors. The hydrobromic acid vapors are removed therein as described above while the natural gas feed and higher molecular weight hydrocarbons are conveyed to the dehydration and product recovery unit where the gas and liquid phases are separated and recovered. The gas stream of recycle and feed gas resulting from the dehydration and product recovery unit, which comprises residual process gases and the natural gas feed, is conveyed to the alkane bromination reactor while water is removed from the higher molecular weight hydrocarbons in the dehydration and product recovery unit to obtain the desirable hydrocarbon liquid products. In this manner, the process illustrated in FIG. 8 produces liquid hydrocarbon products from lower molecular weight hydrocarbons.

A specific embodiment of the process generally depicted in FIG. 8 is described hereafter with reference FIG. 9. A gas stream containing lower molecular weight alkanes, which is a mixture of a feed gas and a recycle gas at a pressure in the range of about 1 bar to about 30 bar, is conveyed via line 62 into line 25. The gas stream mixes further with dry liquid bromine being transported via line 25 by pump 24. The gas stream and dry liquid bromine pass through heat exchanger 26 wherein the liquid bromine is vaporized to dry bromine vapor. The resulting mixture of lower molecular weight alkanes from the gas stream and dry bromine vapor is fed to first reactor 30. The molar ratio of lower molecular weight alkanes to dry bromine vapor in the mixture introduced into first reactor 30 is preferably in excess of 2.5:1. First reactor 30 has an inlet pre-heater zone 28 which heats the mixture to a reaction initiation temperature in the range of about 250° C. to about 400° C.

The lower molecular weight alkanes react exothermically with the dry bromine vapor in first reactor 30 at a relatively low temperature in the range of about 250° C. to about 600° C. and at a pressure in the range of about 1 bar to about 30 bar to produce gaseous alkyl bromides and hydrobromic acid vapors. The upper limit of the operating temperature range is greater than the upper limit of the reaction initiation temperature range to which the feed mixture is heated due to the exothermic nature of the bromination reaction. In the case where the lower molecular weight alkane is methane, methyl bromide is formed in accordance with the following general reaction:

CH₄(g)+Br₂(g)→CH₃Br(g)+HBr(g)

This reaction occurs with a significantly high degree of selectivity to methyl bromide. Furthermore, selectivity to the mono-halogenated methyl bromide increases using a methane to bromine ratio of about 4.5:1 relative to the selectivity obtained using smaller methane to bromine ratios. Small amounts of dibromomethane and tribromomethane are also formed in the bromination reaction. Higher alkanes, such as ethane, propane and butane, are also readily brominated resulting in mono and multiple brominated species such as ethyl bromides, propyl bromides and butyl bromides. If an alkane to bromine ratio of significantly less than about 2.5 to 1 is utilized, a lower selectivity to methyl bromide occurs and significant formation of undesirable carbon soot is observed.

The dry bromine vapor that is fed into first reactor 30 is substantially water-free. It has been discovered that elimination of substantially all water vapor from the bromination step in first reactor 30 substantially eliminates the formation of unwanted carbon dioxide, thereby increasing the selectivity of alkane bromination to alkyl bromides and eliminating the large amount of waste heat generated in the formation of carbon dioxide from alkanes.

The effluent from first reactor 30, which contains alkyl bromides and hydrobromic acid, is withdrawn via line 31 and partially cooled in heat exchanger 32 before being conveyed to a second reactor 34. The temperature to which the effluent is partially cooled in heat exchanger 32 is in the range of about 150° C. to about 350° C. when it is desired to convert the alkyl bromides to higher molecular weight hydrocarbons in second reactor 34 or in the range of about 150° C. to about 450° C. when it is desired to convert the alkyl bromides to olefins in second reactor 34. The alkyl bromides are reacted exothermically in second reactor 34 over a fixed bed 33 of crystalline alumino-silicate catalyst. The temperature and pressure employed in second reactor 34 as well as the specific crystalline alumino-silicate catalyst determine the actual product(s) formed in second reactor 34.

The crystalline alumino-silicate catalyst in fixed bed 33 is preferably a zeolite catalyst and most preferably a ZSM-5 zeolite catalyst when it is desired to form higher molecular weight hydrocarbons. Although the zeolite catalyst is preferably in the hydrogen, sodium or magnesium form, the zeolite may also be modified by ion exchange with other alkali metal cations, such as Li, Na, K or Cs, with alkali-earth metal cations, such as Mg, Ca, Sr or Ba, or with transition metal cations, such as Ni, Mn, V, W, or to the hydrogen form. Other zeolite catalysts having varying pore sizes and acidities, which are synthesized by varying the alumina-to-silica ratio may be used in second reactor 34 as will be evident to a skilled artisan.

When it is desired to form olefins in second reactor 34, the crystalline alumino-silicate catalyst in fixed bed 33 is preferably a zeolite catalyst and most preferably an X type or Y type zeolite catalyst. A preferred zeolite is 10 X or Y type zeolite, although other zeolites with differing pore sizes and acidities, which are synthesized by varying the alumina-to-silica ratio may be used in the process as will be evident to a skilled artisan. Although the zeolite catalyst is preferably used in a protonic form, a sodium form or a mixed protonic/sodium form, the zeolite may also be modified by ion exchange with other alkali metal cations, such as Li, K or Cs, with alkali-earth metal cations, such as Mg, Ca, Sr or Ba, or with transition metal cations, such as Ni, Mn, V, W, or to the hydrogen form. These various alternative cations have an effect of shifting reaction selectivity. Other zeolite catalysts having varying pore sizes and acidities, which are synthesized by varying the alumina-to-silica ratio, may be used in second reactor 34 as will be evident to a skilled artisan.

The temperature at which second reactor 34 is operated is an important parameter in determining the selectivity of the reaction to higher molecular weight or to olefins.

Where a catalyst is selected to form higher molecular weight hydrocarbons in second reactor 34, it is preferred to operate second reactor 34 at a temperature within the range of about 150° to 450° C. Temperatures above about 300° C. in second reactor 34 result in increased yields of light hydrocarbons, such as undesirable methane, whereas lower temperatures increase yields of heavier molecular weight hydrocarbon products. At the low end of the temperature range, for example, with methyl bromide reacting over ZSM-5 zeolite at temperatures as low as 150° C., methyl bromide conversion on the order of 20% is noted with a high selectivity toward C₅+ products. When the alkyl bromide reaction is carried out over the preferred zeolite ZSM-5 catalyst, cyclization reactions also occur such that C₇+ fractions are composed primarily of substituted aromatics.

At increasing temperatures approaching 300° C., methyl bromide conversion increases towards 90% or greater. However, selectivity towards C₅+ products decreases and selectivity towards lighter products, particularly undesirable methane, increases. Surprisingly very little ethane or C₂-C₃ olefin components are formed. At temperatures approaching 450° C. almost complete conversion of methyl bromide to methane occurs.

In the optimum operating temperature range between about 300° C. and 400° C., a small amount of carbon will build up on the catalyst over time during operation as a byproduct of the reaction, which causes a decline in catalyst activity over a range of hours, up to hundreds of hours, depending on the reaction conditions and the composition of the feed gas. It is believed that higher reaction temperatures above about 400° C. associated with the formation of methane favor the thermal cracking of alkyl bromides and formation of carbon or coke and, hence, an increase in the rate of deactivation of the catalyst. Conversely, temperatures at the lower end of the range, particularly below about 300° C., may also contribute to coking due to a reduced rate of desorption of heavier products from the catalyst. Hence, operating temperatures within the range of about 150° C. to about 450° C., but preferably in the range of about 300° C. to about 400° C. in second reactor 34 balance increased selectivity of the desired C₅+ products and lower rates of deactivation due to carbon formation against higher conversion per pass, which minimizes the quantity of catalyst, recycle rates and equipment size required.

Where a catalyst is selected to form olefins in second reactor 34, it is preferred to operate second reactor 34 at a temperature within the range of about 250° C. to 500° C. Temperatures above about 450° C. in second reactor 34 can result in increased yields of light hydrocarbons, such as undesirable methane, and also deposition of coke, whereas lower temperatures increase yields of ethylene, propylene, butylene and heavier molecular weight hydrocarbon products. When the alkyl bromide reaction is carried out over the preferred 10 X zeolite catalyst, it is believed that cyclization reactions also occur such that C₇+ fractions contain substantial substituted aromatics.

At increasing temperatures approaching 400° C., it is believed that methyl bromide conversion increases towards 90% or greater. However, selectivity towards C₅+ products decreases and selectivity towards lighter products, particularly olefins, increases. At temperatures exceeding 550° C., it is believed that a high conversion of methyl bromide to methane and carbonaceous coke occurs.

In the preferred operating temperature range between about 300° C. and 450° C., a lesser amount of coke will likely build up on the catalyst over time during operation as a byproduct of the reaction. It is believed that higher reaction temperatures above about 400° C., associated with the formation of methane, favor the thermal cracking of alkyl bromides and formation of carbon or coke and, hence, an increase in the rate of deactivation of the catalyst. Conversely, temperatures at the lower end of the range, particularly below about 300° C., may also contribute to coking due to a reduced rate of desorption of heavier products from the catalyst. Hence, operating temperatures within the range of about 250° C. to about 500° C. in second reactor 34, but preferably in the range of about 300° C. to about 450° C. balance increased selectivity of the desired olefins and C₅+ products and lower rates of deactivation due to carbon formation against higher conversion per pass, which minimizes the quantity of catalyst, recycle rates and equipment size required.

The catalyst may be periodically regenerated in situ by isolating second reactor 34 from the normal process flow. Once isolated, second reactor 34 is purged with an inert gas via line 70 at a pressure in a range from about 1 to about 5 bar at an elevated temperature in the range of about 400° C. to about 650° C. to remove unreacted material adsorbed on the catalyst insofar as is practical. The deposited carbon is subsequently oxidized to CO₂ by addition of air or inert gas-diluted oxygen to second reactor 34 via line 70 at a pressure in the range of about 1 bar to about 5 bar at an elevated temperature in the range of about 400° C. to about 650° C. Carbon dioxide and residual air or inert gas are vented from second reactor 34 via line 75 during the regeneration period.

The effluent from second reactor 34, which comprises hydrobromic acid and higher molecular weight hydrocarbons, olefins or mixtures thereof, is withdrawn via line 35 and cooled to a temperature in the range of 0° C. to about 100° C. in exchanger 36. The cooled effluent in line 35 is combined with vapor effluent in line 12 from hydrocarbon stripper 47, which contains feed gas and residual higher molecular weight hydrocarbons stripped-out by contact with the feed gas in hydrocarbon stripper 47. The resulting combined vapor mixture is passed to a scrubber 38 and contacted with a concentrated aqueous partially-oxidized metal bromide salt solution, which is transported to scrubber 38 via line 41.

The concentrated aqueous partially-oxidized metal bromide salt solution contains metal hydroxide, metal oxide, metal oxy-bromide or mixtures of these species. The preferred metal of the bromide salt is Fe(III), Cu(II) or Zn(II), or mixtures thereof, which are less expensive and readily oxidize at lower temperatures in the range of about 120° C. to about 180° C., thereby allowing the use of glass-lined or fluoropolymer-lined equipment. However, Co(II), Ni(II), Mn(II), V(II), Cr(II) or other transition-metals, which form oxidizable bromide salts, may also be used in the process. Alternatively, alkaline-earth metals which also form oxidizable bromide salts, such as Ca(II) or Mg(II) may be used. Hydrobromic acid is dissolved in the aqueous solution and neutralized by the metal hydroxide, metal oxide, metal oxy-bromide or mixtures of these species to yield metal bromide salt in solution and water which are removed from scrubber 38 via line 44. Any liquid hydrocarbons condensed in scrubber 38 may be skimmed and withdrawn in line 37 and added to liquid hydrocarbons exiting a product recovery unit 52 in line 54.

The residual vapor phase, which contains olefins, higher molecular weight hydrocarbons or mixtures thereof, is removed from scrubber 38 as effluent and conveyed to dehydrator 50 via line 39 to remove substantially all water from the vapor stream via line 53. The dried vapor stream, which contains olefins, higher molecular weight hydrocarbons or mixtures thereof, is conveyed to product recovery unit 52 via line 51 to where olefins, the C₅+ gasoline-range hydrocarbon fraction or mixtures thereof are recovered as a liquid product via line 54. Any conventional method of dehydration and liquids recovery, such as solid-bed desiccant adsorption followed by refrigerated condensation, cryogenic expansion, or circulating absorption oil or other solvent, as is used to process natural gas or refinery gas streams and/or to recover olefinic hydrocarbons within the purview of a skilled artisan may be employed for this operation.

The residual vapor effluent from product recovery unit 52 is split into a purge stream 57, which may be utilized as fuel for the process, and a recycled residual vapor stream in line 62, which is compressed via compressor 58. The recycled residual vapor discharged from compressor 58 is split into two fractions. A first fraction, which is equal to at least 2.5 times the feed gas molar volume, is transported via line 62, combined with dry liquid bromine, conveyed by pump 24, heated in exchanger 26 to vaporize the bromine and fed into first reactor 30. The second fraction is drawn off of line 62 via line 63 which is regulated by control valve 60 at a rate sufficient to dilute the alkyl bromide concentration to second reactor 34 and absorb the heat of reaction. As such, second reactor 34 is maintained at the selected operating temperature, preferably in the range of about 300° C. to about 450° C., which maximizes conversion versus selectivity and minimizes the rate of catalyst deactivation due to the deposition of carbon. In sum, the dilution provided by the recycled vapor effluent permits controlled selectivity of bromination in first reactor 30 and controlled moderation of the temperature in second reactor 34.

Water containing metal bromide salt in solution, which is removed from scrubber 38 via line 44, is passed to hydrocarbon stripper 47 wherein residual dissolved hydrocarbons are stripped from this aqueous phase by contact with incoming feed gas transported via line 11. The stripped aqueous solution is transported from hydrocarbon stripper 47 via line 65, cooled to a temperature in the range of about 0° C. to about 70° C. in heat exchanger 46 and passed to absorber 48 wherein residual bromine is recovered from vent stream in line 67. The aqueous solution effluent from adsorber 48 is transported via line 49 to a heat exchanger 40, preheated to a temperature in the range of about 100° C. to about 600° C., and most preferably in the range of about 120° C. to about 180° C., and passed to third reactor 16.

Oxygen or air is delivered to a bromine stripper 14 via line 10 by blower or compressor 13 at a pressure in the range of about ambient to about 5 bar to strip residual bromine from water. Water is removed from stripper 14 in line 64 and combined with water stream 53 from dehydrator 50 to form water effluent stream in line 56 which is removed from the process. The oxygen or air leaving bromine stripper 14 is fed via line 15 to reactor 16 which operates at a pressure in the range of about ambient to about 5 bar and at a temperature in the range of about 100° C. to about 600° C., but most preferably in the range of about 120° C. to about 180° C. The oxygen or air oxidizes an aqueous metal bromide salt solution in reactor 16 which yields elemental bromine and metal hydroxide, metal oxide, metal oxy-bromide or mixtures of these species. As stated above, although Co(II), Ni(II), Mn(II), V(II), Cr(II) or other transition-metals which form oxidizable bromide salts can be used, the preferred metal of the bromide salt is Fe(III), Cu(II), or Zn(II), or mixtures thereof. These are less expensive and readily oxidize at lower temperatures in the range of about 120° C. to about 180° C., which should allow the use of glass-lined or fluoropolymer-lined equipment. Alternatively alkaline-earth metals which also form oxidizable bromide salts, such as Ca(II) or Mg(II), could be used.

Hydrobromic acid reacts with the metal hydroxide, metal oxide, metal oxy-bromide or mixtures of these species so formed to once again yield the metal bromide salt and water. Heat exchanger 18 in second reactor 16 supplies heat to vaporize water and bromine. Thus, it is believed that the overall reactions result in the net oxidation of hydrobromic acid produced in first reactor 30 and second reactor 34 to elemental bromine and steam in the liquid phase. The reactions are catalyzed by the metal bromide/metal oxide or metal hydroxide operating in a catalytic cycle.

In the case where the metal bromide is Fe(III) Br₃, the reactions are believed to be:

Fe(+3a)+6Br(−a)+3H(+a)+3/2O₂(g)=3Br₂(g)+Fe(OH)₃ 1)

3HBr(g)+H₂O=3H(+a)+3Br(−a)+H₂O 2)

3H(+a)+3Br(−a)+Fe(OH)₃=Fe(+3a)+3Br(−a)+3H₂O 3)

In the case where the metal bromide is CU(II)Br₂, the reactions are believed to be:

4Cu(+2a)+8Br(−a)+3H₂O+3/2O₂(g)=3Br₂(g)+CuBr₂.3Cu(OH)₂ 1)

6HBr(g)+H₂O=6H(+a)+6Br(−a)+H₂O 2)

6H(+a)+6Br(−a)+CuBr₂.3Cu(OH)₂=4Cu(+2a)+8Br(−a)+6H₂O 3)

The elemental bromine and water and any residual oxygen (and/or nitrogen if air is utilized as the oxidant) leaving as vapor from the outlet of third reactor 16 via line 19 are cooled in condenser 20 at a temperature in the range of about 0° C. to about 70° C. and a pressure in the range of about ambient to 5 bar to condense the bromine and water and passed to three-phase separator 22. Since liquid water has a limited solubility for bromine, on the order of about 3% by weight, any additional bromine which is condensed forms a separate, denser liquid bromine phase in three-phase separator 22. The liquid bromine phase, however, has a notably lower solubility for water, on the order of less than 0.1%. Thus, a substantially dry bromine vapor can be easily obtained by condensing liquid bromine and water, decanting the water by simple physical separation and subsequently re-vaporizing liquid bromine.

Liquid bromine is pumped in line 25 from three-phase separator 22 via pump 24 to a pressure sufficient to mix with vapor stream 62. Thus, bromine is recovered and recycled within the process. The residual oxygen or nitrogen and any residual bromine vapor which is not condensed exits three-phase separator 22 and is passed via line 23 to bromine scrubber 48, wherein residual bromine is recovered by solution into and by reaction with reduced metal bromides in the aqueous metal bromide solution stream 65. Water is removed from separator 22 via line 27 and introduced into stripper 14.

It is readily apparent to a skilled artisan that the above-described process for converting gaseous alkanes to liquid hydrocarbons shown in FIG. 9 can be adapted to incorporate the method of the present invention. Integration of the method of the present invention into the gaseous alkane conversion process is effected by substituting bromine stripper 14, reactor 16 and their cooperative components shown in FIG. 9, which perform the function of converting hydrogen bromide produced in second reactor 34 to elemental bromine and returning it to first reactor 30, with the system of FIG. 2 or 3 for performing the same function. In particular, hydrogen bromide (i.e., hydrobromic acid) contained in the vapor phase effluent exiting second reactor 34 via line 35 of FIG. 9 is separated from the higher molecular weight hydrocarbons, olefins or mixtures thereof, preferably upstream of scrubber 38.

The resulting gaseous hydrogen bromide stream is conveyed to feed gas line to 412 of system 410 or 500 of FIG. 2 or 3, respectively, with or without appropriate pretreatment steps as needed or desired, which may include heating, cooling, expanding, compressing, concentrating, diluting, drying, introducing additives, or the like. After converting the hydrogen bromide to elemental bromine in system 410 or 500 as described above and shown in FIGS. 2 and 3, respectively, the elemental bromine in elemental bromine product recovery line 477 of system 410 or 500 is returned to line 25 in the process of FIG. 9 for appropriate pretreatment, if any, as needed or desired and reinjection into first reactor 30.

In another embodiment described with reference to FIG. 10, a gas stream containing lower molecular weight alkanes, which is a mixture of a feed gas and a recycle gas at a pressure in the range of about 1 bar to about 30 bar, is conveyed via line 162 and mixed further with dry liquid bromine being transported via pump 124. The gas stream and dry liquid bromine pass through heat exchanger 126 wherein the liquid bromine is vaporized to dry bromine vapor. The resulting mixture of lower molecular weight alkanes from the gas stream and dry bromine vapor is fed to first reactor 130. The molar ratio of lower molecular weight alkanes to dry bromine vapor in the mixture introduced into first reactor 130 is preferably in excess of 2.5:1. First reactor 130 has an inlet pre-heater zone 128 which heats the mixture to a reaction initiation temperature in the range of about 250° C. to about 400° C.

The lower molecular weight alkanes react exothermically with the dry bromine vapor in first reactor 130 at a relatively low temperature in the range of about 250° C. to about 600° C. and at a pressure in the range of about 1 bar to about 30 bar to produce gaseous alkyl bromides and hydrobromic acid vapors. The upper limit of the operating temperature range in first reactor 130 is greater than the upper limit of the reaction initiation temperature range due to the exothermic nature of the bromination reaction. In the case where the lower molecular weight alkane is methane, methyl bromide is formed in accordance with the following general reaction:

CH₄(g)+Br₂(g)→CH₃Br(g)+HBr(g)

This reaction occurs with a significantly high degree of selectivity to methyl bromide. Furthermore, selectivity to the mono-halogenated methyl bromide increases using a methane to bromine ratio of about 4.5:1. Small amounts of dibromomethane and tribromomethane are also formed in the bromination reaction. Higher alkanes, such as ethane, propane and butane, are also readily brominated resulting in mono and multiple brominated species such as ethyl bromides, propyl bromides and butyl bromides. If an alkane to bromine ratio of significantly less than about 2.5 to 1 is utilized, a lower selectivity to methyl bromide occurs and significant formation of undesirable carbon soot is observed.

The dry bromine vapor that is fed into first reactor 130 is preferably substantially water-free. It has been discovered that elimination of substantially all water vapor from the bromination step in first reactor 130 substantially eliminates the formation of unwanted carbon dioxide, thereby increasing the selectivity of alkane bromination to alkyl bromides and eliminating the large amount of waste heat generated in the formation of carbon dioxide from alkanes.

The effluent from first reactor 130, which contains alkyl bromides and hydrobromic acid, is withdrawn via line 131 and partially cooled in heat exchanger 132 before being conveyed to a second reactor 134. The temperature to which the effluent is partially cooled in heat exchanger 132 is in the range of about 150° C. to about 350° C. when it is desired to convert the alkyl bromides to higher molecular weight hydrocarbons in second reactor 134 or in the range of about 150° C. to about 450° C. when it is desired to convert the alkyl bromides to olefins in second reactor 134. The alkyl bromides are reacted exothermically in second reactor 134 over a fixed bed 133 of crystalline alumino-silicate catalyst. The temperature and pressure employed in second reactor 134, as well as the crystalline alumino-silicate catalyst, determine the actual product(s) formed in second reactor 134.

The crystalline alumino-silicate catalyst in fixed bed 133 is preferably a zeolite catalyst and most preferably a ZSM-5 zeolite catalyst when it is desired to form higher molecular weight hydrocarbons. Although the zeolite catalyst is preferably in the hydrogen, sodium or magnesium form, the zeolite may also be modified by ion exchange with other alkali metal cations, such as Li, Na, K or Cs, with alkali-earth metal cations, such as Mg, Ca, Sr or Ba, or with transition metal cations, such as Ni, Mn, V, W, or to the hydrogen form. Other zeolite catalysts having varying pore sizes and acidities, which are synthesized by varying the alumina-to-silica ratio may be used in second reactor 134 as will be evident to a skilled artisan.

When it is desired to form olefins from the reaction of alkyl bromides in second reactor 134, the crystalline alumino-silicate catalyst employed in second reactor 134 is preferably a zeolite catalyst, and most preferably an X type or Y type zeolite catalyst. A preferred zeolite is 10 X or Y type zeolite, although other zeolites with differing pore sizes and acidities, which are synthesized by varying the alumina-to-silica ratio may be used in the process as will be evident to a skilled artisan. Although the zeolite catalyst is preferably used in a protonic form, a sodium form or a mixed protonic/sodium form, the zeolite may also be modified by ion exchange with other alkali metal cations, such as Li, K or Cs, with alkali-earth metal cations, such as Mg, Ca, Sr or Ba, or with transition metal cations, such as Ni, Mn, V, W, or to the hydrogen form. These various alternative cations have an effect of shifting reaction selectivity. Other zeolite catalysts having varying pore sizes and acidities, which are synthesized by varying the alumina-to-silica ratio, may be used in second reactor 134 as will be evident to a skilled artisan.

The temperature at which second reactor 134 is operated is an important parameter in determining the selectivity of the reaction to higher molecular weight hydrocarbons or to olefins.

Where a catalyst is selected to form higher molecular weight hydrocarbons in second reactor 134, it is preferred to operate second reactor 134 at a temperature within the range of about 150° to 450° C. Temperatures above about 300° C. in second reactor 134 result in increased yields of light hydrocarbons, such as undesirable methane, whereas lower temperatures increase yields of heavier molecular weight hydrocarbon products. At the low end of the temperature range, for example, with methyl bromide reacting over ZSM-5 zeolite at temperatures as low as 150° C., methyl bromide conversion on the order of 20% is noted with a high selectivity toward C₅+ products. When the alkyl bromide reaction is carried out over the preferred zeolite ZSM-5 catalyst, cyclization reactions also occur such that C₇+ fractions are composed primarily of substituted aromatics.

At increasing temperatures approaching 300° C., methyl bromide conversion increases towards 90% or greater. However, selectivity towards C₅+ products decreases and selectivity towards lighter products, particularly undesirable methane, increases. Surprisingly very little ethane or C₂-C₃ olefin components are formed. At temperatures approaching 450° C. almost complete conversion of methyl bromide to methane occurs.

In the optimum operating temperature range between about 300° C. and 400° C., a small amount of carbon will build up on the catalyst over time during operation as a byproduct of the reaction, which causes a decline in catalyst activity over a range of hours, up to hundreds of hours, depending on the reaction conditions and the composition of the feed gas. It is believed that higher reaction temperatures above about 400° C. associated with the formation of methane favor the thermal cracking of alkyl bromides and formation of carbon or coke and, hence, an increase in the rate of deactivation of the catalyst. Conversely, temperatures at the lower end of the range, particularly below about 300° C., may also contribute to coking due to a reduced rate of desorption of heavier products from the catalyst. Hence, operating temperatures within the range of about 150° C. to about 450° C., but preferably in the range of about 300° C. to about 400° C. in second reactor 134 balance increased selectivity of the desired C₅+ products and lower rates of deactivation due to carbon formation against higher conversion per pass, which minimizes the quantity of catalyst, recycle rates and equipment size required.

Where a catalyst is selected to form olefins in second reactor 134, it is preferred to operate second reactor 134 at a temperature within the range of about 250° C. to 500° C. Temperatures above about 450° C. in second reactor 134 can result in increased yields of light hydrocarbons, such as undesirable methane, and also deposition of coke, whereas lower temperatures increase yields of ethylene, propylene, butylene and heavier molecular weight hydrocarbon products. When the alkyl bromide reaction is carried out over the preferred 10 X zeolite catalyst, it is believed that cyclization reactions also occur such that C₇+ fractions contain substantial substituted aromatics.

At increasing temperatures approaching 400° C., it is believed that methyl bromide conversion increases towards 90% or greater. However, selectivity towards C₅+ products decreases and selectivity towards lighter products, particularly olefins, increases. At temperatures exceeding 550° C., it is believed that a high conversion of methyl bromide to methane and carbonaceous coke occurs.

In the preferred operating temperature range between about 300° C. and 450° C., a lesser amount of coke will likely build up on the catalyst over time during operation as a byproduct of the reaction. It is believed that higher reaction temperatures above about 400° C., associated with the formation of methane, favor the thermal cracking of alkyl bromides and formation of carbon or coke and, hence, an increase in the rate of deactivation of the catalyst. Conversely, temperatures at the lower end of the range, particularly below about 300° C., may also contribute to coking due to a reduced rate of desorption of heavier products from the catalyst. Hence, operating temperatures within the range of about 250° C. to about 500° C. in second reactor 134, but preferably in the range of about 300° C. to about 450° C. balance increased selectivity of the desired olefins and C₅+ products and lower rates of deactivation due to carbon formation against higher conversion per pass, which minimizes the quantity of catalyst, recycle rates and equipment size required.

The catalyst may be periodically regenerated in situ by isolating second reactor 134 from the normal process flow. Once isolated, second reactor 134 is purged with an inert gas via line 170 at a pressure in a range from about 1 to about 5 bar at an elevated temperature in the range of about 400° C. to about 650° C. to remove unreacted material adsorbed on the catalyst insofar as is practical. The deposited carbon is subsequently oxidized to CO₂ by addition of air or inert gas-diluted oxygen to second reactor 134 via line 170 at a pressure in the range of about 1 bar to about 5 bar at an elevated temperature in the range of about 400° C. to about 650° C. Carbon dioxide and residual air or inert gas are vented from second reactor 134 via line 175 during the regeneration period.

The effluent, which comprises hydrobromic acid and higher molecular weight hydrocarbons, olefins or mixtures thereof, is withdrawn from second reactor 134 via line 135, cooled in exchanger 36 to a temperature in the range of 0° C. to about 100° C. and combined with vapor effluent from hydrocarbon stripper 147 in line 112. The resulting mixture is passed to a scrubber 138 and contacted with a stripped recirculated water which has been transported to scrubber 138 via line 164 by any suitable means, such as pump 143, after the stripped recirculated water has been cooled in heat exchanger 155 to a temperature in the range of about 0° C. to about 50° C.

Any liquid hydrocarbon product condensed in scrubber 138 is skimmed, withdrawn as stream 137 and added to liquid hydrocarbon product 154. Hydrobromic acid is dissolved in the aqueous solution in scrubber 138, removed from scrubber 138 via line 144 and conveyed to hydrocarbon stripper 147. Residual hydrocarbons dissolved in the aqueous solution are stripped-out in hydrocarbon stripper 147 by contact with feed gas 111. The stripped aqueous phase from hydrocarbon stripper 147 is cooled in heat exchanger 146 to a temperature in the range of about 0° C. to about 50° C. and conveyed to absorber 148 via line 165 where residual bromine is recovered from vent stream 167.

The residual vapor phase, which contains olefins, higher molecular weight hydrocarbons or mixtures thereof, is removed from scrubber 138 as effluent and conveyed to dehydrator 150 via line 139 to remove substantially all water from the vapor stream via line 153. The dried vapor stream, which contains olefins, higher molecular weight hydrocarbons or mixtures thereof, is conveyed to product recovery unit 152 via line 151 to recover olefins, the C₅+ gasoline range hydrocarbon fraction or mixtures thereof as a liquid product in line 154. Any conventional method of dehydration and liquids recovery within the purview of a skilled artisan, such as solid-bed desiccant adsorption followed by refrigerated condensation, cryogenic expansion, or circulating absorption oil or other solvent, as is used to process natural gas or refinery gas streams and/or to recover olefinic hydrocarbons, may be employed for this operation.

The residual vapor effluent from product recovery unit 152 is split into a purge stream 157, which may be utilized as fuel for the process, and a recycled residual vapor stream in line 162, which is compressed via compressor 158. The recycled residual vapor discharged from compressor 158 is split into two fractions. A first fraction, which is equal to at least 2.5 times the feed gas molar volume, is transported via line 162, combined with dry liquid bromine, conveyed by pump 124, heated in exchanger 126 to vaporize the bromine and fed into first reactor 130. The second fraction is drawn off line 162 via line 163, which is regulated by control valve 160 at a rate sufficient to dilute the alkyl bromide concentration to second reactor 134 and absorb the heat of reaction. As such, second reactor 134 is maintained at the selected operating temperature, preferably in the range of about 300° C. to about 450° C., which maximizes conversion versus selectivity and minimizes the rate of catalyst deactivation due to the deposition of carbon. In sum, the dilution provided by the recycled vapor effluent permits controlled selectivity of bromination in first reactor 130 and controlled moderation of the temperature in second reactor 134.

Oxygen, oxygen-enriched air or air 110 is delivered to bromine stripper 114 via blower or compressor 113 at a pressure in the range of about ambient to about 5 bar and strips residual bromine from water. The stripped water is discharged from stripper 114 via line 164 and is divided into two portions. The first portion of stripped water is recycled to the process via line 164 while the second portion is removed from the process via line 156. The first portion of stripped water is cooled in heat exchanger 155 to a temperature in the range of about 20° C. to about 50° C. and maintained by any suitable means, such as pump 143, at a pressure sufficient to enter scrubber 138. The relative volume of the first portion is selected such that the hydrobromic acid solution effluent removed from scrubber 138 via line 144 has a concentration in the range from about 10% to about 50% by weight hydrobromic acid, and more preferably in the range of about 30% to about 48% by weight. This minimizes the amount of water which must be vaporized in exchanger 141 and preheater 119 and minimizes the vapor pressure of HBr over the resulting hydrobromic acid.

The dissolved hydrobromic acid in the aqueous solution effluent from adsorber 148 is transported via line 149 and combined with the oxygen, oxygen-enriched air or air leaving bromine stripper 114 via line 115. The combined aqueous solution effluent and oxygen, oxygen-enriched air or air is passed to a first side of heat exchanger 141, through preheater 119 where the mixture is preheated to a temperature in the range of about 100° C. to about 600° C., and most preferably in the range of about 120° C. to about 250° C., and on to third reactor 117 which is an oxidation reactor containing a metal bromide salt or metal oxide. The preferred metal of the bromide salt or metal oxide is Fe(III), Cu(II) or Zn(II), although Co(II), Ni(II), Mn(II), V(II), Cr(II) or other transition-metals which form oxidizable bromide salts can be used. Alternatively, alkaline-earth metals which also form oxidizable bromide salts, such as Ca(II) or Mg(II) could be used.

The metal bromide salt in oxidation reactor 117 can be in the form of a concentrated aqueous solution, but preferably the concentrated aqueous salt solution is imbibed into a porous, high surface area, acid resistant inert support such as a silica gel. More preferably, the oxide form of the metal, which is in a concentration range of 10 to 20% by weight, is deposited on an inert support such as alumina with a specific surface area in the range of 50 to 200 m²/g.

The oxidation reactor 117 operates at a pressure in the range of about ambient to about 5 bar and at a temperature in the range of about 100° C. to 600° C., and most preferably in the range of about 130° C. to 350° C. Within these operating ranges, the metal bromide is oxidized by oxygen, yielding elemental bromine and metal hydroxide, metal oxide or metal oxy-bromide species. Elemental bromine and metal oxides are yielded in the case of a supported metal bromide salt or in the case where the oxidation reactor 117 is operated at higher temperatures and lower pressures at which water primarily exists as a vapor. In any case, the hydrobromic acid reacts with the metal hydroxide, metal oxy-bromide or metal oxide species and is neutralized, restoring the metal. It is believed that the overall reaction results in the net oxidation of hydrobromic acid produced in first reactor 130 and second reactor 134 to elemental bromine and steam. The reactions are catalyzed by the metal bromide/metal oxide or metal hydroxide operating in a catalytic cycle.

In the case where the metal bromide is Fe(III)Br₂ in an aqueous solution within a pressure and temperature range in which water may exist as a liquid, the reactions are believed to be:

Fe(+3a)+6Br(−a)+3H(+a)+3/2O₂(g)=3Br₂(g)+Fe(OH)3 1)

3HBr(g)+H₂O=3H(+a)+3Br(−a)+H₂O 2)

3H(+a)+3Br(−a)+Fe(OH)3=Fe(+3a)+3Br(−a)+3H₂O 3)

In the case where the metal bromide is CU(II)Br₂ in an aqueous solution and within a pressure and temperature range in which water may exist as a liquid, the reactions are believed to be:

4Cu(+2a)+8Br(−a)+3H₂O+3/2O₂(g)=3Br₂(g)+CuBr₂.3Cu(OH)₂ 1)

6HBr(g)+H₂O=6H(+a)+6Br(−a)+H₂O 2)

6H(+a)+6Br(−a)+CuBr₂.3Cu(OH)₂=4Cu(+2a)+8Br(−a)+6H₂O 3)

In the case where the metal bromide is Cu(II)Br₂ supported on an inert support and at higher temperature and lower pressure conditions at which water primarily exists as a vapor, the reactions are believed to be:

2Cu(II)Br₂=2Cu(I)Br+Br₂(g) 1)

2Cu(I)Br+O₂(g)=Br₂(g)+2Cu(II)O 2)

2HBr(g)+Cu(II)O=Cu(II)Br₂+H₂O(g) 3)

The elemental bromine and water and any residual oxygen (and/or nitrogen if air is utilized as the oxidant) leaving as vapor from the outlet of oxidation reactor 117 are cooled in the second side of exchanger 141 and condenser 120 to a temperature in the range of about 0° C. to about 70° C. wherein the bromine and water are condensed and passed to three-phase separator 122. Since liquid water has a limited solubility for bromine, on the order of about 3% by weight, any additional bromine which is condensed forms a separate, denser liquid bromine phase in three-phase separator 122. The liquid bromine phase, however, has a notably lower solubility for water, on the order of less than 0.1%. Thus, a substantially dry bromine vapor can be easily obtained by condensing liquid bromine and water, decanting the water by simple physical separation and subsequently re-vaporizing liquid bromine. It is important to operate at conditions that result in the near complete reaction of HBr so as to avoid significant residual HBr in the condensed liquid bromine and water. HBr increases the miscibility of bromine in the aqueous phase, and at sufficiently high concentrations, results in a single ternary liquid phase.

Liquid bromine is pumped in line 125 from three-phase separator 122 via pump 124 to a pressure sufficient to mix with vapor stream 162. Thus the bromine is recovered and recycled within the process. The residual air, oxygen-enriched air or oxygen and any bromine vapor which is not condensed exits three-phase separator 122 and is passed via line 123 to bromine scrubber 148, wherein residual bromine is recovered by dissolution into the hydrobromic acid solution stream conveyed to scrubber 148 via line 165. Water is removed from the three-phase separator 122 via line 129 and passed to stripper 114.

The elemental bromine vapor and steam are condensed and easily separated in the liquid phase by simple physical separation yielding substantially dry bromine. The absence of significant water allows selective bromination of alkanes without production of CO₂ and the subsequent efficient and selective reactions of alkyl bromides to primarily C₂ to C₄ olefins, heavier products the C₅+ fraction of which contains substantial branched alkanes and substituted aromatics, or mixtures thereof. Byproduct hydrobromic acid vapor from the bromination reaction in first reactor 130 and the subsequent reaction in second reactor 134 is readily dissolved into an aqueous phase and neutralized by the metal hydroxide or metal oxide species resulting from oxidation of the metal bromide.

It is readily apparent to a skilled artisan that the above-described process for converting gaseous alkanes to liquid hydrocarbons shown in FIG. 10 can be adapted to incorporate the method of the present invention. Integration of the method of the present invention into the gaseous alkane conversion process is effected by substituting bromine stripper 114, oxidation reactor 117 and their cooperative components shown in FIG. 10, which perform the function of converting hydrogen bromide produced in second reactor 134 to elemental bromine and returning it to first reactor 130, with the system of FIG. 2 or 3 for performing the same function. In particular, hydrogen bromide (i.e., hydrobromic acid) contained in the vapor phase effluent exiting second reactor 134 via line 135 of FIG. 10 is separated from the higher molecular weight hydrocarbons, olefins or mixtures thereof, preferably upstream of scrubber 138.

The resulting gaseous hydrogen bromide stream is conveyed to feed gas line to 412 of system 410 or 500 of FIG. 2 or 3, respectively, with or without appropriate pretreatment steps as needed or desired, which may include heating, cooling, expanding, compressing, concentrating, diluting, drying, introducing additives, or the like. After converting the hydrogen bromide to elemental bromine in system 410 or 500 as described above and shown in FIGS. 2 and 3, respectively, the elemental bromine in elemental bromine product recovery line 477 of system 410 or 500 is returned to line 125 in the process of FIG. 10 for appropriate pretreatment, if any, as needed or desired and injection into first reactor 130.

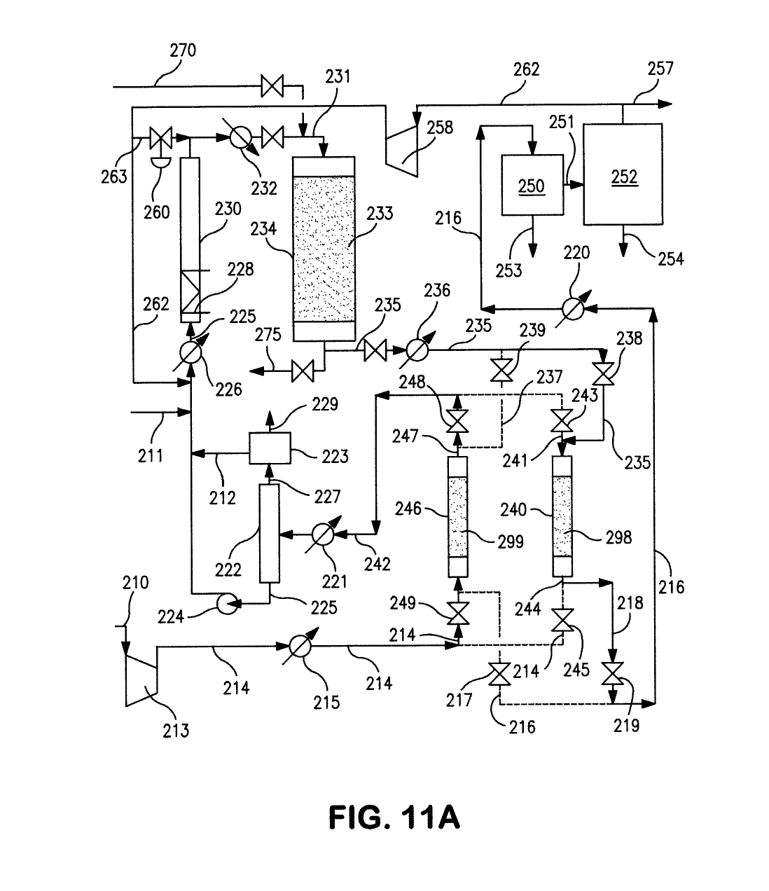

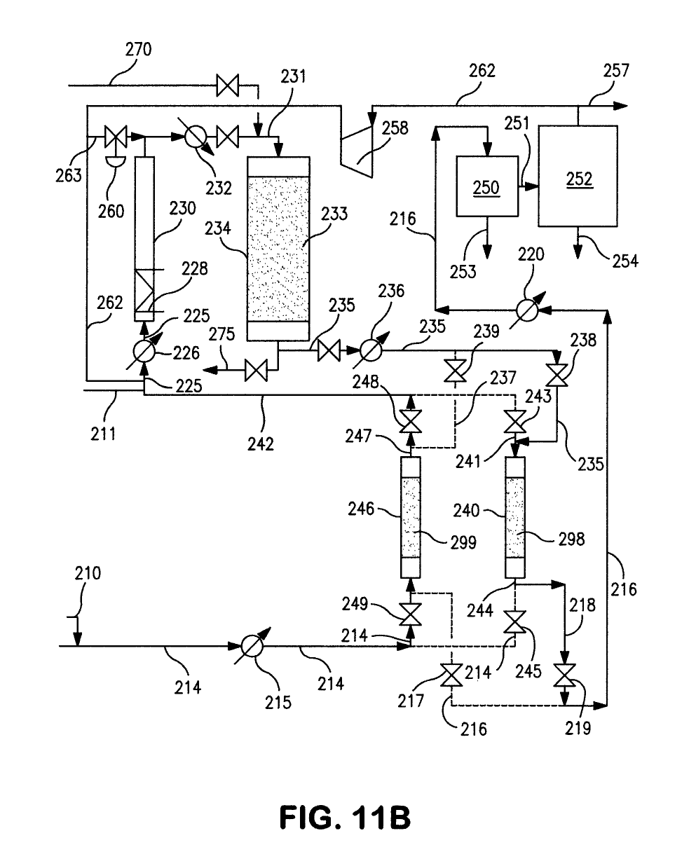

In accordance with another embodiment illustrated in FIG. 11A, the alkyl bromination and alkyl bromide conversion stages are operated in a substantially similar manner to those corresponding stages described with respect to FIGS. 9 and 10 above. More particularly, a gas stream containing lower molecular weight alkanes and comprised of a feed gas and a recycle gas mixture at a pressure in the range of about 1 bar to about 30 bar is conveyed via lines 262 and 211, respectively, and mixed with dry liquid bromine in line 225. The resulting mixture is transported via pump 224 and passed to heat exchanger 226 wherein the liquid bromine is vaporized. The mixture of lower molecular weight alkanes from the gas stream and dry bromine vapor is fed to a first reactor 230. The molar ratio of lower molecular weight alkanes to dry bromine vapor in the mixture introduced into first reactor 230 is preferably in excess of 2.5:1.

First reactor 230 has an inlet pre-heater zone 228 which heats the mixture to a reaction initiation temperature in the range of 250° C. to 400° C. The lower molecular weight alkanes react exothermically with the dry bromine vapor in first reactor 230 at a relatively low temperature in the range of about 250° C. to about 600° C. and at a pressure in the range of about 1 bar to about 30 bar to produce gaseous alkyl bromides and hydrobromic acid vapors. The upper limit of the operating temperature range is greater than the upper limit of the reaction initiation temperature range due to the exothermic nature of the bromination reaction. In the case where the lower molecular weight alkane is methane, methyl bromide is formed in accordance with the following general reaction:

CH₄(g)+Br₂(g)→CH₃Br(g)+HBr(g)

This reaction occurs with a significantly high degree of selectivity to methyl bromide. Furthermore, selectivity to the mono-halogenated methyl bromide increases using a methane to bromine ratio of about 4.5:1. Small amounts of dibromomethane and tribromomethane are also formed in the bromination reaction. Higher alkanes, such as ethane, propane and butane, are also readily brominated resulting in mono and multiple brominated species such as ethyl bromides, propyl bromides and butyl bromides. If an alkane to bromine ratio of significantly less than 2.5 to 1 is utilized, substantially lower selectivity to methyl bromide occurs and significant formation of undesirable carbon soot is observed.

The dry bromine vapor that is fed into first reactor 230 is substantially water-free. Elimination of substantially all water vapor from the bromination step in first reactor 230 substantially eliminates the formation of unwanted carbon dioxide, thereby increasing the selectivity of alkane bromination to alkyl bromides and eliminating the large amount of waste heat generated in the formation of carbon dioxide from alkanes.

The effluent from first reactor 230, which contains alkyl bromides and hydrobromic acid, is withdrawn via line 231 and partially cooled in heat exchanger 232 before being conveyed to a second reactor 234. The temperature to which the effluent is partially cooled in heat exchanger 232 is in the range of about 150° C. to about 350° C. when it is desired to convert the alkyl bromides to higher molecular weight hydrocarbons in second reactor 234 or in the range of about 150° C. to about 450° C. when it is desired to convert the alkyl bromides to olefins in second reactor 234. The alkyl bromides are reacted exothermically in second reactor 234 over a fixed bed 233 of crystalline alumino-silicate catalyst. The temperature and pressure employed in second reactor 234, as well as the specific crystalline alumino-silicate catalyst, determine the product formed in second reactor 234.

The crystalline alumino-silicate catalyst employed in fixed bed 233 is preferably a zeolite catalyst and most preferably a ZSM-5 zeolite catalyst when it is desired to form higher molecular weight hydrocarbons. Although the zeolite catalyst is preferably in the hydrogen, sodium or magnesium form, the zeolite may also be modified by ion exchange with other alkali metal cations, such as Li, Na, K or Cs, with alkali-earth metal cations, such as Mg, Ca, Sr or Ba, or with transition metal cations, such as Ni, Mn, V, W, or to the hydrogen form. Other zeolite catalysts having varying pore sizes and acidities, which are synthesized by varying the alumina-to-silica ratio, may be used in second reactor 234 as will be evident to a skilled artisan.

When it is desired to form olefins from the reaction of alkyl bromides in second reactor 234, the crystalline alumino-silicate catalyst employed in second reactor 234 is preferably a zeolite catalyst and most preferably an X type or Y type zeolite catalyst. A preferred zeolite is 10 X or Y type zeolite, although other zeolites with differing pore sizes and acidities, which are synthesized by varying the alumina-to-silica ratio, may be used in the process as will be evident to a skilled artisan. Although the zeolite catalyst is preferably used in a protonic form, a sodium form or a mixed protonic/sodium form, the zeolite may also be modified by ion exchange with other alkali metal cations, such as Li, K or Cs, with alkali-earth metal cations, such as Mg, Ca, Sr or Ba, or with transition metal cations, such as Ni, Mn, V, W, or to the hydrogen form. These various alternative cations have an effect of shifting reaction selectivity. Other zeolite catalysts having varying pore sizes and acidities, which are synthesized by varying the alumina-to-silica ratio, may be used in second reactor 234 as will be evident to a skilled artisan.

The temperature at which second reactor 234 is operated is an important parameter in determining the selectivity of the reaction to higher molecular weight, or to olefins.

Where a catalyst is selected to form higher molecular weight hydrocarbons in second reactor 234, it is preferred to operate second reactor 234 at a temperature within the range of about 150° to 450° C. Temperatures above about 300° C. in second reactor 234 result in increased yields of light hydrocarbons, such as undesirable methane, whereas lower temperatures increase yields of heavier molecular weight hydrocarbon products. At the low end of the temperature range, for example, with methyl bromide reacting over ZSM-5 zeolite at temperatures as low as 150° C., methyl bromide conversion on the order of 20% is noted with a high selectivity toward C₅+ products. When the alkyl bromide reaction is carried out over the preferred zeolite ZSM-5 catalyst, cyclization reactions also occur such that C₇+ fractions are composed primarily of substituted aromatics.

At increasing temperatures approaching 300° C., methyl bromide conversion increases towards 90% or greater. However, selectivity towards C₅+ products decreases and selectivity towards lighter products, particularly undesirable methane, increases. Surprisingly very little ethane or C₂-C₃ olefin components are formed. At temperatures approaching 450° C. almost complete conversion of methyl bromide to methane occurs.

In the optimum operating temperature range between about 300° C. and 400° C., a small amount of carbon will build up on the catalyst over time during operation as a byproduct of the reaction, which causes a decline in catalyst activity over a range of hours, up to hundreds of hours, depending on the reaction conditions and the composition of the feed gas. It is believed that higher reaction temperatures above about 400° C. associated with the formation of methane favor the thermal cracking of alkyl bromides and formation of carbon or coke and, hence, an increase in the rate of deactivation of the catalyst. Conversely, temperatures at the lower end of the range, particularly below about 300° C., may also contribute to coking due to a reduced rate of desorption of heavier products from the catalyst. Hence, operating temperatures within the range of about 150° C. to about 450° C., but preferably in the range of about 300° C. to about 400° C. in second reactor 234 balance increased selectivity of the desired C₅+ products and lower rates of deactivation due to carbon formation against higher conversion per pass, which minimizes the quantity of catalyst, recycle rates and equipment size required.

Where a catalyst is selected to form olefins in second reactor 234, it is preferred to operate second reactor 234 at a temperature within the range of about 250° C. to 500° C. Temperatures above about 450° C. in second reactor 234 can result in increased yields of light hydrocarbons, such as undesirable methane, and also deposition of coke, whereas lower temperatures increase yields of ethylene, propylene, butylene and heavier molecular weight hydrocarbon products. When the alkyl bromide reaction is carried out over the preferred 10 X zeolite catalyst, it is believed that cyclization reactions also occur such that C₇+ fractions contain substantial substituted aromatics. At increasing temperatures approaching 400° C., it is believed that methyl bromide conversion increases towards 90% or greater. However, selectivity towards C₅+ products decreases and selectivity towards lighter products, particularly olefins, increases. At temperatures exceeding 550° C., it is believed that a high conversion of methyl bromide to methane and carbonaceous coke occurs.

In the preferred operating temperature range between about 300° C. and 450° C., a lesser amount of coke will likely build up on the catalyst over time during operation as a byproduct of the reaction. It is believed that higher reaction temperatures above about 400° C., associated with the formation of methane, favor the thermal cracking of alkyl bromides and formation of carbon or coke and, hence, an increase in the rate of deactivation of the catalyst. Conversely, temperatures at the lower end of the range, particularly below about 300° C., may also contribute to coking due to a reduced rate of desorption of heavier products from the catalyst. Hence, operating temperatures within the range of about 250° C. to about 500° C. in second reactor 234, but preferably in the range of about 300° C. to about 450° C. balance increased selectivity of the desired olefins and C₅+ products and lower rates of deactivation due to carbon formation against higher conversion per pass, which minimizes the quantity of catalyst, recycle rates and equipment size required.

The catalyst may be periodically regenerated in situ by isolating second reactor 234 from the normal process flow. Once isolated, second reactor 234 is purged with an inert gas via line 270 at a pressure in a range from about 1 to about 5 bar at an elevated temperature in the range of about 400° C. to about 650° C. to remove unreacted material adsorbed on the catalyst insofar as is practical. The deposited carbon is subsequently oxidized to CO₂ by addition of air or inert gas-diluted oxygen to second reactor 234 via line 270 at a pressure in the range of about 1 bar to about 5 bar at an elevated temperature in the range of about 400° C. to about 650° C. Carbon dioxide and residual air or inert gas are vented from second reactor 234 via line 275 during the regeneration period.

The effluent, which comprises hydrobromic acid and higher molecular weight hydrocarbons, olefins or mixtures thereof, is withdrawn from second reactor 234 via line 235 and cooled in exchanger 236 to a temperature in the range of about 100° C. to about 600° C. As illustrated in FIG. 11A, the cooled effluent is transported via lines 235 and 241 with valve 238 in the opened position and valves 239 and 243 in the closed position and introduced into a reactor 240 containing a bed 298 of a solid phase metal oxide. The metal of the metal oxide is selected form magnesium (Mg), calcium (Ca), vanadium (V), chromium (Cr), manganese (Mn), iron (Fe), cobalt (Co), nickel (Ni), copper (Cu), zinc (Zn), or tin (Sn).

The metal is selected for the impact of its physical and thermodynamic properties relative to the desired temperature of operation and also for potential environmental and health impacts and cost. Magnesium, copper and/or iron are preferably employed as the metal, with magnesium being the most preferred. These metals have the property of not only forming oxides, but bromide salts as well, with the reactions being reversible in a temperature range of less than about 500° C. The solid metal oxide is preferably immobilized on a suitable attrition-resistant support, for example a synthetic amorphous silica, such as Davicat Grade 57, manufactured by Davison Catalysts of Columbia, Md., or more preferably, an alumina support with a specific surface area of about 50 to 200 m²/g.

Hydrobromic acid is reacted with the metal oxide in reactor 240 at temperatures below about 600° C. and preferably between about 100° C. to about 500° C. in accordance with the following general formula, wherein M represents the metal:

2HBr+MO→MBr₂+H₂O

The steam resulting from this reaction is transported together with olefins and/or the high molecular hydrocarbons in lines 244, 218 and 216 via opened valve 219 to heat exchanger 220, wherein the mixture is cooled to a temperature in the range of about 0° C. to about 70° C. This cooled mixture is forwarded to dehydrator 250 to remove substantially all water from the gas stream via line 253. The dried gas stream containing olefins, higher molecular weight hydrocarbons or mixtures thereof is passed to product recovery unit 252 via line 251 to recover olefins, the C₅+ fraction, or mixtures thereof in line 254 as a liquid product. Any conventional method of dehydration and liquids recovery within the purview of a skilled artisan, such as solid-bed desiccant adsorption followed by refrigerated condensation, cryogenic expansion, or circulating absorption oil or other solvent, as is used to process natural gas or refinery gas streams and/or to recover olefinic hydrocarbons, may be employed for this operation.

The residual vapor effluent from product recovery unit 252 is split into a purge stream 257, which may be utilized as fuel for the process, and a recycled residual vapor, which is compressed via compressor 258. The recycled residual vapor discharged from compressor 258 is split into two fractions. A first fraction, which is equal to at least 1.5 times the feed gas volume, is transported via line 262, combined with the liquid bromine and feed gas conveyed in line 225, passed to heat exchanger 226 wherein the liquid bromine is vaporized, and fed into first reactor 230 in a manner described above. The second fraction is drawn off line 262 via line 263, which is regulated by control valve 260, at a rate sufficient to dilute the alkyl bromide concentration to second reactor 234 and absorb the heat of reaction. As such, reactor 234 is maintained at the selected operating temperature, preferably in the range of about 300° C. to about 450° C., which maximizes conversion versus selectivity and minimizes the rate of catalyst deactivation due to the deposition of carbon. In sum, the dilution provided by the recycled vapor effluent permits controlled selectivity of bromination in first reactor 230 and controlled moderation of the temperature in second reactor 234.

Oxygen, oxygen-enriched air or air 210 is delivered to a reactor 246 via blower or compressor 213, line 214 and valve 249 at a pressure in the range of about ambient to about 10 bar. The oxygen, oxygen-enriched air or air is preheated in heat exchanger 215 to a temperature in the range of about 100° C. to about 500° C. before entering reactor 246 which contains a bed 299 of a solid phase metal bromide. Oxygen reacts with the metal bromide in accordance with the following general reaction, wherein M represents the metal:

MBr₂+1/2O₂→MO+Br₂

A dry, substantially HBr-free bromine vapor is produced in this manner, thereby eliminating the need for subsequent separation of water or hydrobromic acid from the liquid bromine. Reactor 246 is operated below 600° C., and more preferably between about 300° C. to about 500° C. The resultant bromine vapor is transported from reactor 246 via line 247, valve 248 and line 242 to heat exchanger or condenser 221 where the bromine is condensed into a liquid. The liquid bromine is transported via line 242 to separator 222 wherein liquid bromine is removed via line 225 and transported to heat exchanger 226 and first reactor 230 by any suitable means, such as pump 224.

The residual air or unreacted oxygen is transported from separator 222 via line 227 to a bromine scrubbing unit 223, such as a venturi scrubbing system containing a suitable solvent, or suitable solid adsorbent medium, as selected by a skilled artisan, wherein the remaining bromine is captured. The captured bromine is desorbed from the scrubbing solvent or adsorbent by heating or other suitable means. The recovered bromine is transported via line 212 to line 225. The scrubbed air or oxygen is vented via line 229. In this manner, nitrogen and any other substantially non-reactive components are removed from the system of the process, thereby preventing them from entering the hydrocarbon-containing portion of the process. In addition, loss of bromine to the surrounding environment is avoided.

One advantage of removing the HBr by chemical reaction in accordance with the present embodiment, rather than by simple physical solubility, is the substantially complete scavenging of the HBr to low levels at higher process temperatures. Another distinct advantage is the elimination of water from the bromine removed thereby eliminating the need for separation of bromine and water phases and for stripping of residual bromine from the water phase.

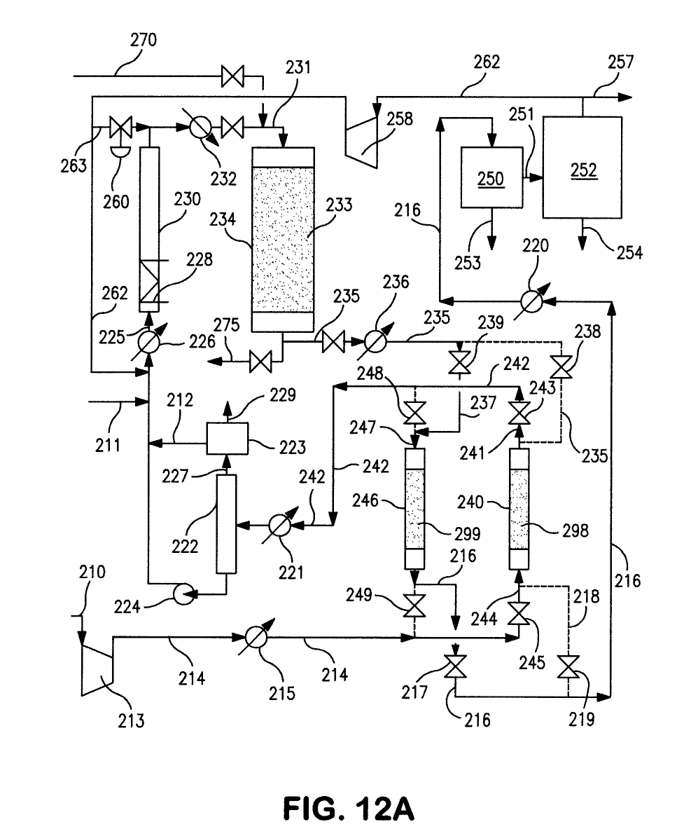

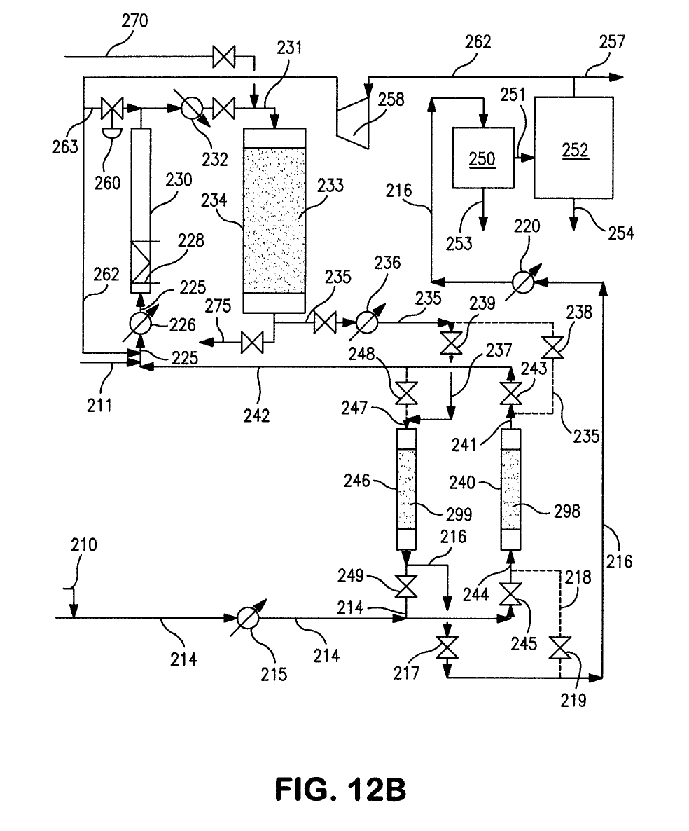

Reactors 240 and 246 may be operated in a cyclic fashion. As illustrated in FIG. 11A, valves 238 and 219 are operated in the open mode to permit hydrobromic acid to be removed from the effluent withdrawn from second reactor 234, while valves 248 and 249 are operated in the open mode to permit air, oxygen-enriched air or oxygen to flow through reactor 246 to oxidize the solid metal bromide contained therein. Once significant conversion of the metal oxide and metal bromide in reactors 240 and 246, respectively, has occurred, valves 248 and 249 are closed. At this point, bed 299 in reactor 246 is a bed of substantially solid metal bromide, while bed 298 in reactor 240 is substantially solid metal oxide. As illustrated in FIG. 12A, valves 245 and 243 are then opened to permit oxygen, oxygen-enriched air or air to flow through reactor 240 to oxidize the solid metal bromide contained therein, while valves 239 and 217 are opened to permit effluent which comprises olefins, the higher molecular weight hydrocarbons and/or hydrobromic acid withdrawn from second reactor 234 to be introduced into reactor 246. The reactors are operated in this manner until significant conversion of the metal oxide and metal bromide in reactors 246 and 240, respectively, has occurred and then the reactors are cycled back to the flow schematic illustrated in FIG. 11A by opening and closing valves as previously discussed.

It is readily apparent to a skilled artisan that the above-described process for converting gaseous alkanes to liquid hydrocarbons shown in FIGS. 11A and 12A can be adapted to incorporate the method of the present invention. Integration of the method of the present invention into the gaseous alkane conversion process is effected by substituting reactors 240 and 246, separator 222, bromine scrubbing unit 223 and their cooperative components shown in FIGS. 11A and 12A, which perform the function of converting hydrogen bromide produced in second reactor 234 to elemental bromine and returning it to first reactor 230, with the system of FIG. 2 or 3 for performing the same function. In particular, hydrogen bromide (i.e., hydrobromic acid) contained in the vapor phase effluent exiting second reactor 234 via line 235 of FIGS. 11A and 12A is separated from the higher molecular weight hydrocarbons, olefins or mixtures thereof.

The resulting gaseous hydrogen bromide stream is conveyed to feed gas line to 412 of system 410 or 500 of FIG. 2 or 3, respectively, with or without appropriate pretreatment steps as needed or desired, which may include heating, cooling, expanding, compressing, concentrating, diluting, drying, introducing additives, or the like. After converting the hydrogen bromide to elemental bromine in system 410 or 500 as described above and shown in FIGS. 2 and 3, respectively, the elemental bromine in elemental bromine product recovery line 477 of system 410 or 500 is returned to line 225 in the process of FIGS. 11A and 12A for appropriate pretreatment, if any, as needed or desired and injection into first reactor 230.

When oxygen is transported via line 210 utilized as the oxidizing gas in reactors 240 and 246, the embodiment illustrated in FIGS. 11A and 12A can be modified such that the bromine vapor produced from either reactor 246 (FIG. 11B) or 240 (FIG. 12B) is transported via lines 242 and 225 directly to first reactor 230. Since oxygen is reactive and will not build up in the system, the need to condense the bromine vapor to a liquid to remove unreactive components, such as nitrogen, is obviated. Compressor 213 is not illustrated in FIGS. 11B and 12B since substantially all commercial sources of oxygen, such as a commercial air separator unit, will provide oxygen to line 210 at the required pressure. If not, a compressor 213 could be utilized to achieve such pressure as will be evident to a skilled artisan.

It is readily apparent to a skilled artisan that the above-described process for converting gaseous alkanes to liquid hydrocarbons shown in FIGS. 11B and 12B can be adapted to incorporate the method of the present invention in substantially the same manner as described above with respect to FIGS. 11A and 12A.

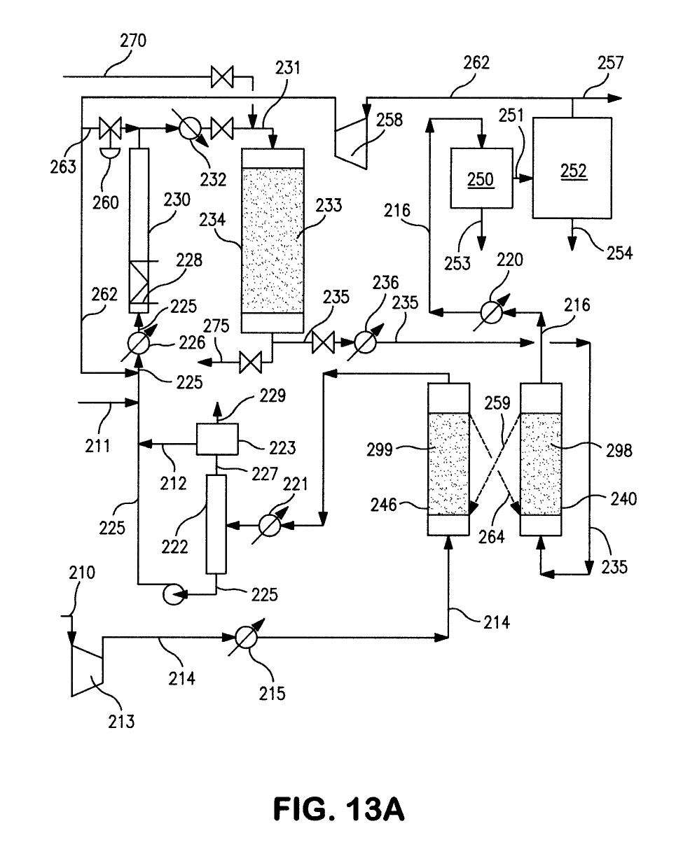

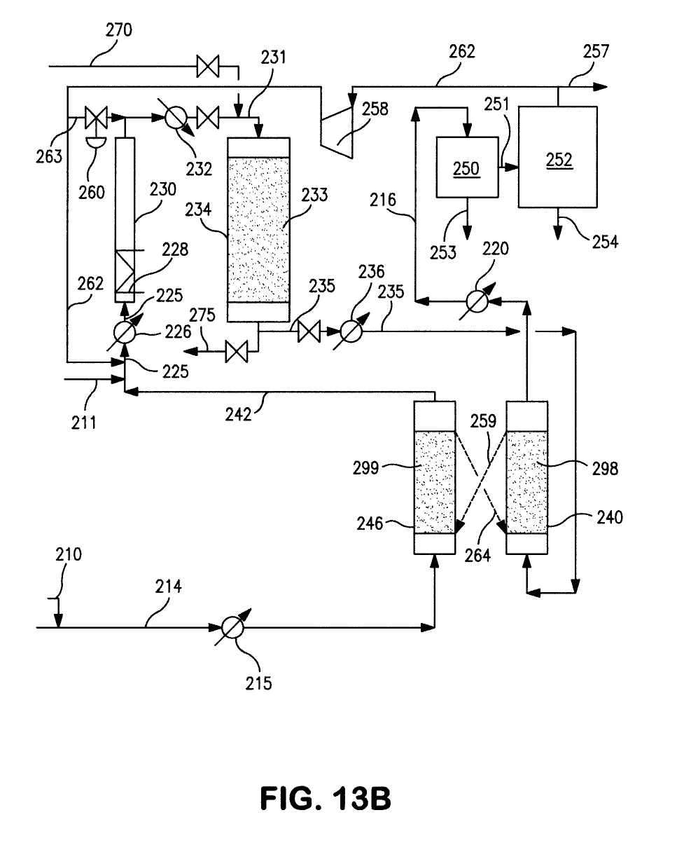

In the embodiment illustrated in FIG. 13A, the beds of solid metal oxide particles and solid metal bromide particles contained in reactors 240 and 246, respectively, are fluidized and are connected in the manner described below to provide for continuous operation of the fluidized beds without the need to provide for equipment, such as valves, to change flow direction to and from each reactor 240 and 246. In accordance with this embodiment, the effluent which comprises olefins, the higher molecular weight hydrocarbons and/or hydrobromic acid is withdrawn from second reactor 234 via line 235, cooled to a temperature in the range of about 100° C. to about 500° C. in exchanger 236, and introduced into the bottom of reactor 240 which contains a bed 298 of solid metal oxide particles.

The flow of introduced fluid induces the particles in bed 298 to move upwardly within reactor 240 as the hydrobromic acid is reacted with the metal oxide in the manner described above with respect to FIG. 11A. At or near the top of bed 298, the particles of bed 298 contain substantially solid metal bromide on the attrition-resistant support due to the substantially complete reaction of the solid metal oxide with hydrobromic acid in reactor 240. Accordingly, the particles of bed 298 are withdrawn from at or near the top of bed 298 of reactor 240 via a weir or cyclone or other conventional means of solid/gas separation, flow by gravity down line 259 and are introduced at or near the bottom of a bed 299 of solid metal bromide particles in reactor 246.

Oxygen, oxygen-enriched air or air in line 210 is delivered to reactor 246 after initially passing through blower or compressor 213 and pressurized to a pressure in the range of about ambient to about 10 bar. The oxygen, oxygen-enriched air or air is also transported via line 214 through heat exchanger 215, wherein the oxygen, oxygen-enriched air or air is preheated to a temperature in the range of about 100° C. to about 500° C., before introduction into reactor 246 below bed 299 of solid phase metal bromide. Oxygen reacts with the metal bromide in the manner described above with respect to FIG. 11A to produce a dry, substantially HBr-free bromine vapor.

The flow of introduced gas induces the particles in bed 299 to flow upwardly within reactor 246 as oxygen reacts with the metal bromide. At or near the top of bed 299, the particles of bed 299 contain substantially solid metal bromide on the attrition-resistant support due to the substantially complete reaction of the solid metal oxide with hydrobromic acid in reactor 240. Accordingly, the particles of bed 299 are withdrawn from at or near the top of bed 299 of reactor 246 via a weir or cyclone or other conventional means of solid/gas separation, flow by gravity down line 264 and are introduced at or near the bottom of bed 298 of solid metal oxide particles in reactor 240. In this manner, reactors 240 and 246 may be operated continuously without changing the parameters of operation.

It is readily apparent to a skilled artisan that the above-described process for converting gaseous alkanes to liquid hydrocarbons shown in FIG. 13A can be adapted to incorporate the method of the present invention. Integration of the method of the present invention into the gaseous alkane conversion process is effected by substituting reactors 240 and 246, separator 222, bromine scrubbing unit 223 and their cooperative components shown in FIG. 13A, which perform the function of converting hydrogen bromide produced in second reactor 234 to elemental bromine and returning it to first reactor 230, with the system of FIG. 2 or 3 for performing the same function. In particular, hydrogen bromide (i.e., hydrobromic acid) contained in the vapor phase effluent exiting second reactor 234 via line 235 of FIG. 13A is separated from the higher molecular weight hydrocarbons, olefins or mixtures thereof.

The resulting gaseous hydrogen bromide stream is conveyed to feed gas line to 412 of system 410 or 500 of FIG. 2 or 3, respectively, with or without appropriate pretreatment steps as needed or desired, which may include heating, cooling, expanding, compressing, concentrating, diluting, drying, introducing additives, or the like. After converting the hydrogen bromide to elemental bromine in system 410 or 500 as described above and shown in FIGS. 2 and 3, respectively, the elemental bromine in elemental bromine product recovery line 477 of system 410 or 500 is returned to line 225 in the process of FIG. 13A for appropriate pretreatment, if any, as needed or desired and injection into first reactor 230.

In the embodiment illustrated in FIG. 13B, oxygen is utilized as the oxidizing gas and is transported via line 210 to reactor 246. Accordingly, the embodiment illustrated in FIG. 13A is modified such that the bromine vapor produced from reactor 246 is transported via lines 242 and 225 directly to first reactor 230. Since oxygen is reactive and will not build up in the system, it is believed that the need to condense the bromine vapor to a liquid to remove unreactive components, such as nitrogen, should be obviated. Compressor 213 is not illustrated in FIG. 13B as substantially all commercial sources of oxygen, such as a commercial air separator unit, will provide oxygen to line 210 at the required pressure. If not, a compressor 213 could be utilized to achieve such pressure as will be evident to a skilled artisan.

It is readily apparent to a skilled artisan that the above-described process for converting gaseous alkanes to liquid hydrocarbons shown in FIG. 13B can be adapted to incorporate the method of the present invention in substantially the same manner as described above with respect to FIG. 13A.

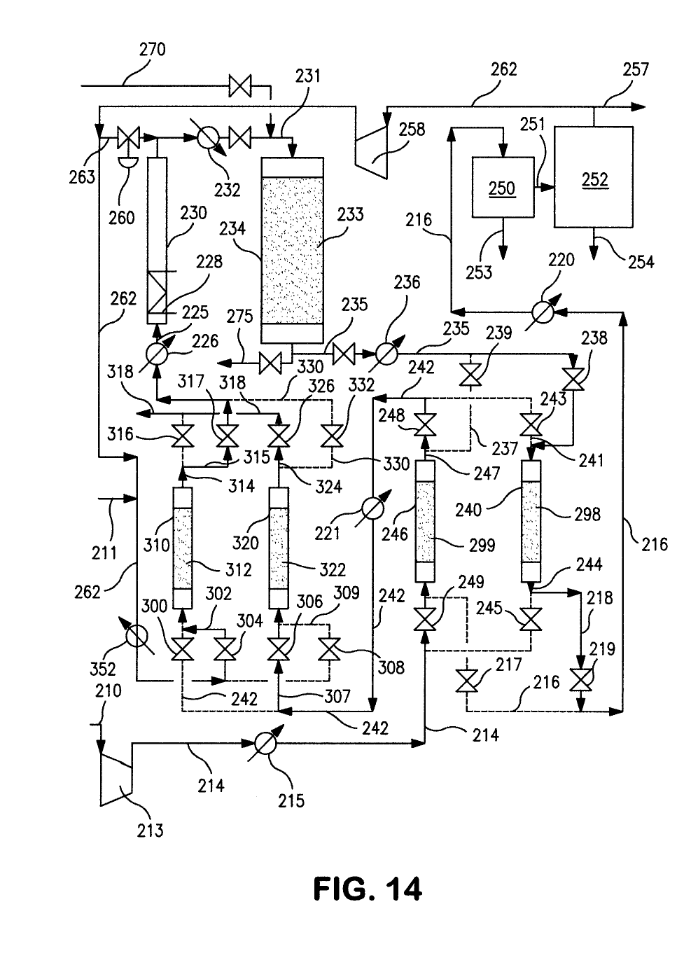

In accordance with another embodiment illustrated in FIG. 14, the alkyl bromination and alkyl bromide conversion stages are operated in a substantially similar manner to those corresponding stages described in detail with respect to FIG. 11A except as discussed below. Residual air or oxygen and bromine vapor emanating from reactor 246 are transported via line 247, valve 248 and line 242 and valve 300 to heat exchanger or condenser 221 wherein the bromine-containing vapor is cooled to a temperature in the range of about 30° C. to about 300° C. The bromine-containing vapor is then transported via line 242 to reactor 320 containing a bed 422 of a solid phase metal bromide in a reduced valence state. The metal of the metal bromide is selected from copper (Cu), iron (Fe), or molybdenum (Mo). The metal is selected based on its physical and thermodynamic properties at the desired temperature of operation and also its potential environmental and health impacts and cost. Copper or iron are preferably employed as the metal, with copper being the most preferred.

The solid metal bromide is preferably immobilized on a suitable attrition-resistant support, for example a synthetic amorphous silica, such as Davicat Grade 57, manufactured by Davison Catalysts of Columbia, Md. More preferably the metal is deposited in oxide form in a range of about 10 to 20 wt % on an alumina support with a specific surface area in the range of about 50 to 200 m²/g, Bromine vapor reacts with the solid phase metal bromide, preferably retained on a suitable attrition-resistant support, in reactor 320 at temperatures below about 300° C. and preferably between about 30° C. to about 200° C. in accordance with the following general formula wherein M² represents the metal:

2M²Brₙ+Br₂→2M²Brₙ₊₁

In this manner, bromine is stored as a second metal bromide, i.e. 2M²Brₙ₊₁, in reactor 320 while the resultant vapor containing residual air or oxygen is vented from reactor 320 via line 324, valve 326 and line 318.

The gas stream in line 262 containing lower molecular weight alkanes, which is a mixture of a feed gas (line 211) and a recycle gas, is conveyed to a reactor 310 via heat exchanger 352, wherein the gas stream is preheated to a temperature in the range of about 150° C. to about 600° C., valve 304 and line 302. Reactor 310 contains a bed 312 of a solid phase metal bromide in an oxidized valence state. The metal of the metal bromide is selected from copper (Cu), iron (Fe), or molybdenum (Mo). The metal is selected based on its physical and thermodynamic properties at the desired temperature of operation and also its potential environmental and health impacts and cost. Copper or iron are preferably employed as the metal, with copper being the most preferred.

The solid metal bromide in an oxidized state is preferably immobilized on a suitable attrition-resistant support, for example a synthetic amorphous silica such as Davicat Grade 57, manufactured by Davison Catalysts of Columbia, Md. More preferably the metal is deposited in an oxidized state in a range of 10 to 20 wt % supported on an alumina support with a specific surface area of about 50 to 200 m²/g. The temperature of the gas stream is from about 150° C. to about 600° C., and preferably from about 200° C. to about 450° C. The temperature of the gas stream thermally decomposes the solid phase metal bromide in an oxidized valence state in reactor 310 to yield elemental bromine vapor and a solid metal bromide in a reduced state in accordance with the following general formula wherein M² represents the metal:

2M²Brₙ₊₁→2M²Brₙ+Br₂

The resultant bromine vapor is transported with the gas stream containing lower molecular weight alkanes via lines 314, 315, valve 317, line 330, heat exchanger 226 into alkyl bromination reactor 230.

Reactors 310 and 320 may operate in a cyclic fashion. As illustrated in FIG. 14, valve 304 is operated in the open mode to permit the gas stream containing lower molecular weight alkanes to be transported to reactor 310, while valve 317 is operated in the open mode to permit this gas stream with bromine vapor that is generated in reactor 310 to be transported to alkyl bromination reactor 230. Likewise, valve 306 is operated in the open mode to permit bromine vapor from reactor 246 to be transported to reactor 320, while valve 326 is operated in the open mode to permit residual air or oxygen to be vented from reactor 320.

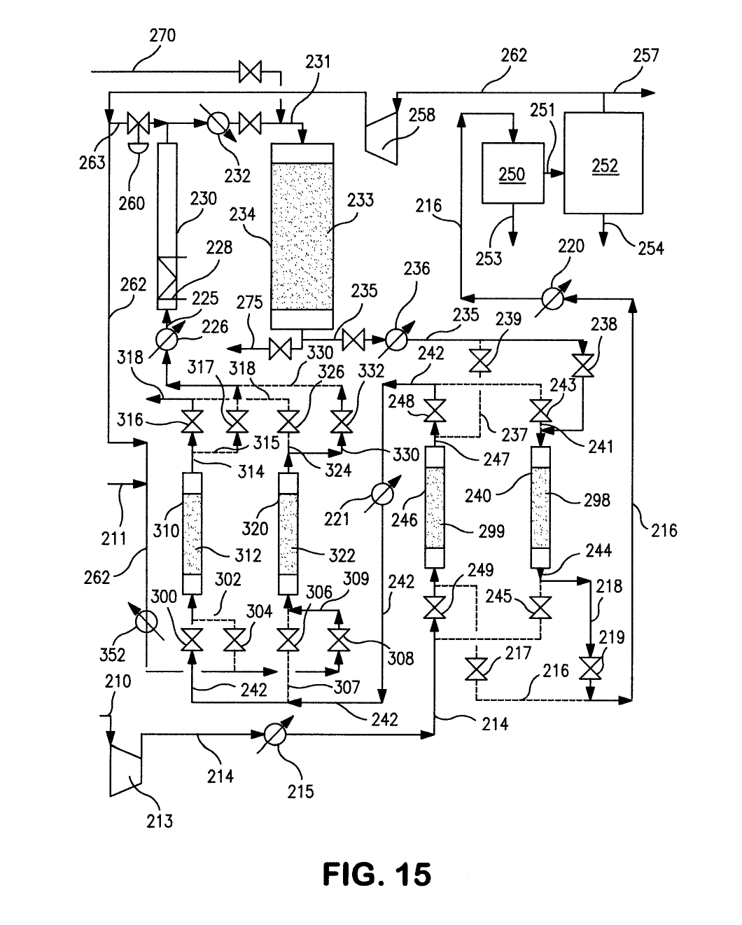

As illustrated in FIG. 15, once significant conversion of the reduced metal bromide and oxidized metal bromide to the corresponding oxidized and reduced states has occurred in reactors 320 and 310, respectively, valves 304, 317, 306, and 326 are closed. At this point, bed 422 in reactor 320 is a bed of substantially metal bromide in an oxidized state, while bed 312 in reactor 310 is substantially metal bromide in a reduced state. When valves 304, 317, 306 and 326 are closed, valves 308 and 332 are opened to permit the gas stream containing lower molecular weight alkanes to be conveyed to reactor 320 via lines 262, heat exchanger 352, wherein gas stream is heated to a range of about 150° C. to about 600° C., valve 308 and line, 309. The solid phase metal bromide in an oxidized valence state is thermally decomposed in reactor 320 to yield elemental bromine vapor and a solid metal bromide in a reduced state.

Valve 332 is also opened to permit the resultant bromine vapor to be transported with the gas stream containing lower molecular weight alkanes via lines 324 and 330 and heat exchanger 226 prior to being introduced into alkyl bromination reactor 230. In addition, valve 300 is opened to permit bromine vapor emanating from reactor 246 to be transported via line 242 through exchanger 221 into reactor 310 wherein the solid phase metal bromide in a reduced valence state reacts with bromine to effectively store bromine as a metal bromide. In addition, valve 316 is opened to permit the resulting gas, which is substantially devoid of bromine to be vented via lines 314 and 318.

The reactors are operated in this manner until significant conversion of the beds of reduced metal bromide and oxidized metal bromide in reactors 310 and 320, respectively, to the corresponding oxidized and reduced states has occurred. Reactors 310 and 320 are then cycled back to the flow schematic illustrated in FIG. 14 by opening and closing valves as previously discussed.

It is readily apparent to a skilled artisan that the above-described process for converting gaseous alkanes to liquid hydrocarbons shown in FIGS. 14 and 15 can be adapted to incorporate the method of the present invention. Integration of the method of the present invention into the gaseous alkane conversion process is effected by substituting reactors 310, 320, 240 and 246, and their cooperative components shown in FIGS. 14 and 15, which perform the function of converting hydrogen bromide produced in second reactor 234 to elemental bromine and returning it to first reactor 230, with the system of FIG. 2 or 3 for performing the same function. In particular, hydrogen bromide (i.e., hydrobromic acid) contained in the vapor phase effluent exiting second reactor 234 via line 235 of FIGS. 14 and 15 is separated from the higher molecular weight hydrocarbons, olefins or mixtures thereof.

The resulting gaseous hydrogen bromide stream is conveyed to feed gas line to 412 of system 410 or 500 of FIG. 2 or 3, respectively, with or without appropriate pretreatment steps as needed or desired, which may include heating, cooling, expanding, compressing, concentrating, diluting, drying, introducing additives, or the like. After converting the hydrogen bromide to elemental bromine in system 410 or 500 as described above and shown in FIGS. 2 and 3, respectively, the elemental bromine in elemental bromine product recovery line 477 of system 410 or 500 is returned to line 330 in the process of FIGS. 14 and 15 for appropriate pretreatment, if any, as needed or desired and injection into first reactor 230.

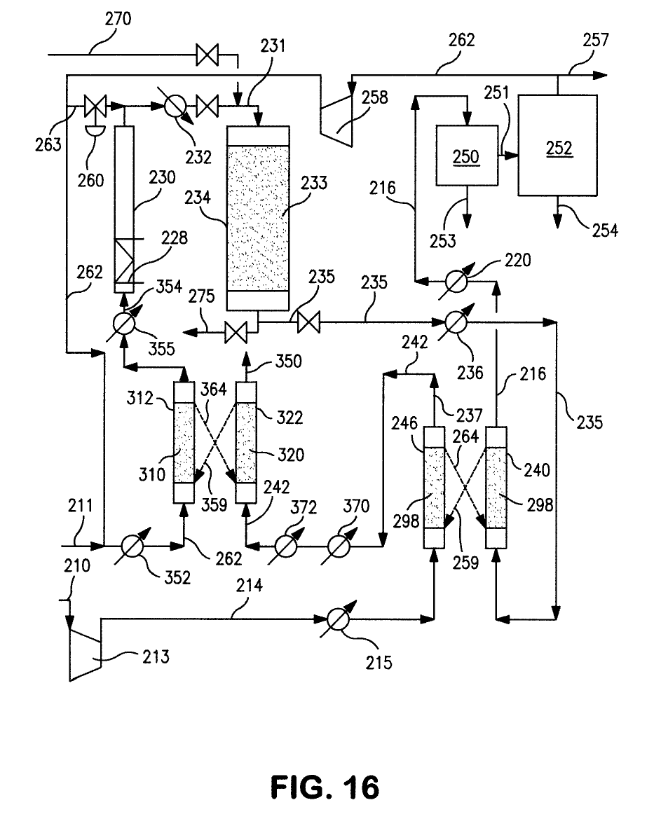

In the embodiment illustrated in FIG. 16, the beds 312 and 322 contained in reactors 310 and 320, respectively, are fluidized and are connected in the manner described below to provide for continuous operation of the beds without the need to provide for equipment, such as valves, to change flow direction to and from each reactor 310 and 320. In accordance with this embodiment, the bromine-containing vapor withdrawn from the reactor 246 via line 242 is cooled to a temperature in the range of about 30° C. to about 300° C. in exchangers 370 and 372, and introduced into the bottom of reactor 320 which contains solid bed 322 in a fluidized state.

The flow of introduced fluid induces the particles in bed 322 to flow upwardly within reactor 320 as the bromine vapor is reacted with the reduced metal bromide entering the bottom of bed 322 in the manner described above with respect to FIG. 14. At or near the top of the bed 322, the particles of bed 322 contain substantially oxidized metal bromide on the attrition-resistant support due to the substantially complete reaction of the reduced metal bromide with bromine vapor in reactor 320. Accordingly, the particles of bed 322 are withdrawn from at or near the top of bed 322 of reactor 320 via a weir, cyclone or other conventional means of solid/gas separation, flow by gravity down line 359 and are introduced at or near the bottom of the bed 312 in reactor 310.

The gas stream in line 262 containing lower molecular weight alkanes, which is a mixture of a feed gas (line 211) and a recycle gas, is conveyed to reactor 310 via heat exchanger 352, wherein the gas stream is preheated to a temperature in the range of about 150° C. to about 600° C., valve 304 and line 302. The heated gas stream is introduced into the bottom of reactor 310 which induces the particles in bed 312 to flow upwardly within reactor 310. The heated gas stream thermally decomposes the solid phase metal bromide in an oxidized valence state entering at or near the bottom of bed 312 to yield elemental bromine vapor and a solid metal bromide in a reduced state. The elemental bromine is withdrawn from reactor 310 via line 354 and exchanger 355 for reintroduction into first reactor 230.

The particles at or near the top of the bed 312 contain substantially reduced solid metal bromide on the attrition-resistant support due to the substantially complete thermal decomposition in reactor 310. The particles are withdrawn at or near the top of the bed 312 of reactor 310 via a weir or cyclone or other conventional means of gas/solid separation and flow by gravity down line 364. The withdrawn particles are introduced at or near the bottom of bed 322 of reactor 310. In this manner, reactors 310 and 320 may be operated continuously without changing the parameters of operation.

It is readily apparent to a skilled artisan that the above-described process for converting gaseous alkanes to liquid hydrocarbons shown in FIG. 16 can be adapted to incorporate the method of the present invention. Integration of the method of the present invention into the gaseous alkane conversion process is effected by substituting reactors 310, 320, 240 and 246, and their cooperative components shown in FIG. 16, which perform the function of converting hydrogen bromide produced in second reactor 234 to elemental bromine and returning it to first reactor 230, with the system of FIG. 2 or 3 for performing the same function. In particular, hydrogen bromide (i.e., hydrobromic acid) contained in the vapor phase effluent exiting second reactor 234 via line 235 of FIG. 16 is separated from the higher molecular weight hydrocarbons, olefins or mixtures thereof.

The resulting gaseous hydrogen bromide stream is conveyed to feed gas line to 412 of system 410 or 500 of FIG. 2 or 3, respectively, with or without appropriate pretreatment steps as needed or desired, which may include heating, cooling, expanding, compressing, concentrating, diluting, drying, introducing additives, or the like. After converting the hydrogen bromide to elemental bromine in system 410 or 500 as described above and shown in FIGS. 2 and 3, respectively, the elemental bromine in elemental bromine product recovery line 477 of system 410 or 500 is returned to line 354 in the process of FIG. 16 for appropriate pretreatment, if any, as needed or desired and injection into first reactor 230.

It is believed that all the above-recited embodiments of the associated upstream process for producing desirable liquid hydrocarbon products are less expensive than other conventional processes since the present process operates at low pressures in the range of about 1 bar to about 30 bar and at relatively low temperatures in the range of about 20° C. to about 600° C. for the gas phase and preferably about 20° C. to about 180° C. for the liquid phase. It is believed that these operating conditions permit the use of less expensive equipment of relatively simple design which are constructed from readily available metal alloys or glass-lined equipment for the gas phase and polymer-lined or glass-lined vessels, piping and pumps for the liquid phase.

It is believed that the present associated upstream process for producing desirable liquid hydrocarbon products is also more efficient because less energy is required for operation and the production of excessive carbon dioxide as an unwanted byproduct is minimized. The process is capable of directly producing a mixed hydrocarbon product containing various molecular-weight components in the liquefied petroleum gas (LPG), olefin and motor gasoline fuels range that have substantial aromatic content, thereby significantly increasing the octane value of the gasoline-range fuel components.

The following examples demonstrate the present associated upstream process for producing desirable liquid hydrocarbon products.

### Example 5

Various mixtures of dry bromine and methane are reacted homogeneously at temperatures in the range of 459° C. to 491° C. at a Gas Hourly Space Velocity (GHSV) of approximately 7200 hr⁻¹. GHSV is defined as the gas flow rate in standard liters per hour divided by the gross reactor catalyst-bed volume, including catalyst-bed porosity in liters. The results of this example indicate that for molar ratios of methane to bromine greater than 4.5:1 selectivity to methyl bromide is in the range of 90 to 95% with near-complete conversion of bromine.

### Example 6

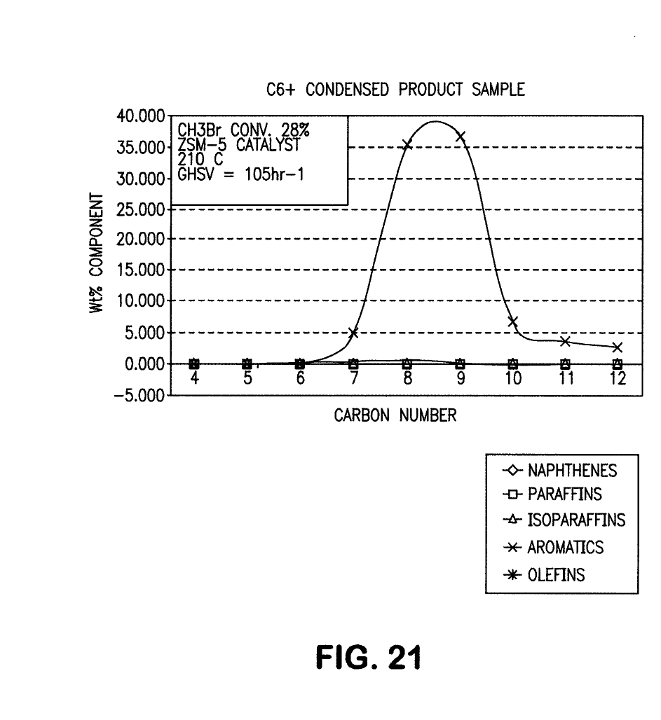

FIG. 20 and FIG. 21 illustrate two exemplary PONA analyses of two C₆+ liquid product samples that are recovered during two test runs with methyl bromide and methane reacting over ZSM-5 zeolite catalyst. These analyses show the substantially aromatic content of the C₆+ fractions produced.

### Example 7

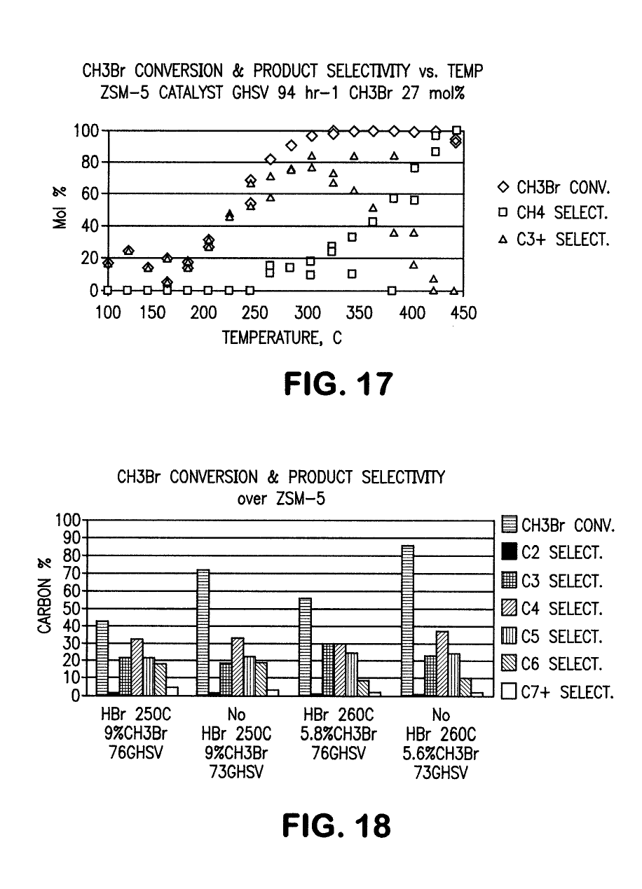

Methyl bromide is reacted over a ZSM-5 zeolite catalyst at a Gas Hourly Space Velocity (GHSV) of approximately 94 hr⁻¹ over a range of temperatures from about 100° C. to about 460° C. at approximately 2 bar pressure. As illustrated in FIG. 17, which is a graph of methyl bromide conversion and product selectivity for the oligomerization reaction as a function of temperature, methyl bromide conversion increases rapidly in the range of about 200° C. to about 350° C. Lower temperatures in the range of about 100° C. to about 250° C. favor selectivity towards higher molecular weight products however conversion is low. Higher temperatures in the range of about 250° C. to about 350° C. show higher conversions in the range of 50% to near 100%, however, increasing selectivity to lower molecular weight products, in particular undesirable methane, is observed. At higher temperatures above 350° C. selectivity to methane rapidly increases. At about 450° C. almost complete conversion to methane occurs.

### Example 8

Methyl bromide, hydrogen bromide and methane are reacted over a ZSM-5 zeolite catalyst at approximately 2 bar pressure at about 250° C. and also at about 260° C. at a GHSV of approximately 76 hr⁻¹. Comparison tests utilizing a mixture of only methyl bromide and methane without hydrogen bromide over the same ZSM-5 catalyst at approximately the same pressure at about 250° C. and at about 260° C. at a GHSV of approximately 73 hr⁻¹ were also run. FIG. 18, which is a graph that illustrates the comparative conversions and selectivities of several example test runs, shows only a very minor effect due to the presence of HBr on product selectivities. Because hydrobromic acid has only a minor effect on conversion and selectivity, it is not necessary to remove the hydrobromic acid generated in the bromination reaction step prior to the conversion reaction of the alkyl bromides, in which additional hydrobromic acid is formed in any case. Thus, the process can be substantially simplified.

### Example 9

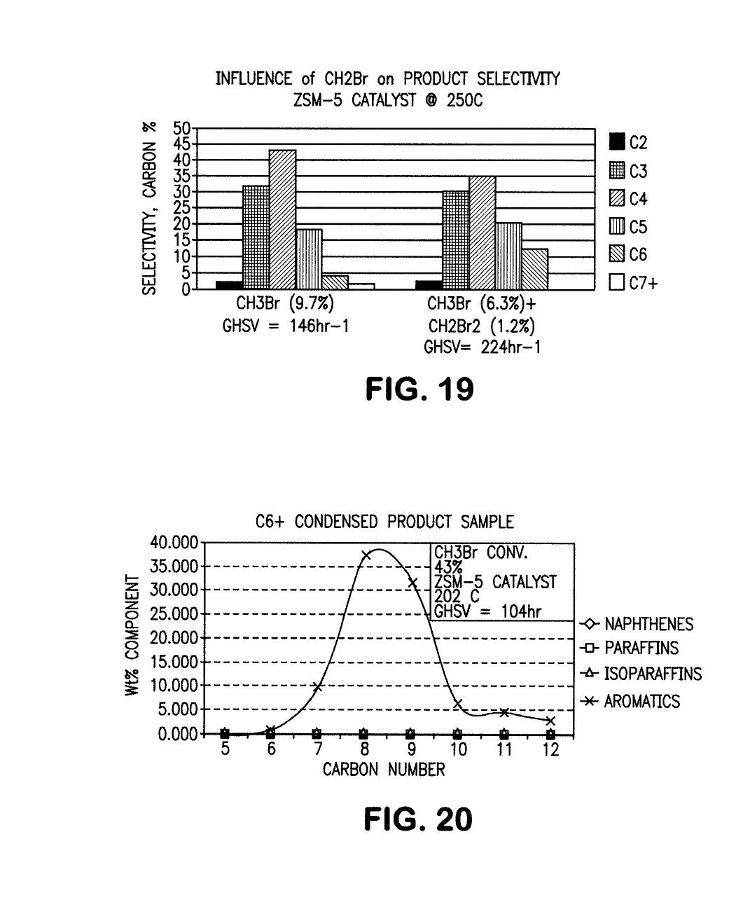

Methyl bromide is reacted over a ZSM-5 zeolite catalyst at 230° C. Dibromomethane is added to the reactor. FIG. 19, which is a graph of product selectivity, indicates that reaction of methyl bromide and dibromomethane results in a shift in selectivity towards C₅+ products versus methyl bromide alone. Thus, these results demonstrate that dibromomethane is also reactive and therefore very high selectivity to bromomethane in the bromination step is not required in the present process. It has been observed, however, that the presence of dibromomethane increases the rate of catalyst deactivation, requiring a higher operating temperature to optimize the tradeoff between selectivity and deactivation rate, as compared to pure methyl bromide.

### Example 10

A mixture of 12.1 mol % methyl bromide and 2.8 mol % propyl bromide in methane are reacted over a ZSM-5 zeolite catalyst at 295° C. and a GHSV of approximately 260 hr⁻¹. A methyl bromide conversion of approximately 86% and a propyl bromide conversion of approximately 98% is observed.

Thus, in accordance with all embodiments of the process set forth above, the metal bromide/metal hydroxide, metal oxy-bromide or metal oxide operates in a catalytic cycle allowing bromine to be easily recycled within the process. The metal bromide is readily oxidized by oxygen, oxygen-enriched air or air either in the aqueous phase or the vapor phase at temperatures in the range of about 100° C. to about 600° C. and most preferably in the range of about 120° C. to about 180° C. to yield elemental bromine vapor and metal hydroxide, metal oxy-bromide or metal oxide. Operation at temperatures below about 180° C. is advantageous, thereby allowing the use of low-cost corrosion-resistant fluoropolymer-lined equipment. Hydrobromic acid is neutralized by reaction with the metal hydroxide or metal oxide yielding steam and the metal bromide.

While the foregoing preferred embodiments of the invention have been described and shown, it is understood that alternatives and modifications, such as those suggested and others, may be made thereto and fall within the scope of the present invention.

We claim:

1. A method for converting hydrogen bromide to elemental bromine comprising the steps of:
thermally oxidizing a portion of an initial hydrogen bromide-rich gas at a thermal oxidation temperature to produce a first fraction of elemental bromine and a remainder of said initial hydrogen bromide-rich gas; and
catalytically oxidizing at least a portion of said remainder of said initial hydrogen bromide-rich gas at a catalytic oxidation temperature to produce a second fraction of elemental bromine.

2. The method of claim 1, wherein said initial hydrogen bromide-rich gas is a substantially dry gas mixture.

3. The method of claim 1, wherein said thermal oxidation temperature is substantially greater than said catalytic oxidation temperature.

4. The method of claim 1, wherein said catalytic oxidation temperature is in a range of about 250° C. to about 345° C.

5. The method of claim 1, wherein thermal oxidation of said portion of said initial hydrogen bromide-rich gas converts about 80% to 99% of total hydrogen bromide in said initial hydrogen bromide-rich gas to elemental bromine.

6. The method of claim 1, wherein thermal oxidation of said portion of said initial hydrogen bromide-rich gas converts about 85% to 95% of total hydrogen bromide in said initial hydrogen bromide-rich gas to elemental bromine.

7. The method of claim 1, wherein catalytic oxidation of said at least a portion of said remainder of said initial hydrogen bromide-rich gas converts about 20% to 1% of total hydrogen bromide in said initial hydrogen bromide-rich gas to elemental bromine.

8. The method of claim 1, wherein catalytic oxidation of said at least a portion of said remainder of said initial hydrogen bromide-rich gas converts about 15% to 5% of total hydrogen bromide in said initial hydrogen bromide-rich gas to elemental bromine.

9. The method of claim 1 further comprising deriving said initial hydrogen bromide-rich gas from a hydrogen bromide-containing gas.

10. The method of claim 9, wherein said hydrogen bromide-containing gas has a lower hydrogen bromide concentration than said initial hydrogen bromide-rich gas.

11. The method of claim 9, wherein said initial hydrogen bromide-rich gas is said hydrogen bromide-containing gas.

12. The method of claim 9, wherein said hydrogen bromide-containing gas is a gaseous mixture containing hydrogen bromide and lower molecular weight hydrocarbons.

13. The method of claim 9, wherein said hydrogen bromide-containing gas is derived from an upstream process.

14. The method of claim 13, wherein said upstream process is an associated process.

15. The method of claim 13, wherein said upstream process is an unrelated process.

16. The method of claim 13, wherein said upstream process is a gaseous alkane conversion process and further wherein gaseous alkanes are converted to liquid hydrocarbons by brominating said gaseous alkanes and catalytically reacting resulting alkyl bromides to form said liquid hydrocarbons.

17. The method of claim 1 further comprising converting gaseous alkanes to liquid hydrocarbons in a gaseous alkane conversion process by brominating said gaseous alkanes and catalytically reacting resulting alkyl bromides to form said liquid hydrocarbons and a hydrogen bromide-containing gas, and deriving said initial hydrogen bromide-rich gas from said hydrogen bromide-containing gas.

18. The method of claim 17 further comprising recycling said first and second fractions of elemental bromine as a feed to said gaseous alkane conversion process.

19. The method of claim 1 further comprising adding an oxidizing gas to said initial hydrogen bromide-rich gas during or before thermally oxidizing said initial hydrogen bromide-rich gas.

20. A method for converting hydrogen bromide to elemental bromine comprising the steps of:
adding an oxidizing gas to an initial hydrogen bromide-rich gas to form a thermal oxidation feed gas, wherein said initial hydrogen bromide-rich gas is a substantially dry gas mixture including hydrogen bromide;
thermally oxidizing a portion of said thermal oxidation feed gas in a thermal oxidation reactor at a thermal oxidation temperature to produce a first fraction of elemental bromine and a remainder of said thermal oxidation feed gas; and
catalytically oxidizing at least a portion of said remainder of said thermal oxidation feed gas in a catalytic reactor at a catalytic oxidation temperature to produce a second fraction of elemental bromine, wherein said thermal oxidation temperature is substantially greater than said catalytic oxidation temperature.

21. The method of claim 20 further comprising recovering said first and second fractions of elemental bromine as an elemental bromine product from a catalytic reactor effluent gas discharged from said catalytic reactor.

22. The method of claim 21 further comprising condensing said catalytic reactor effluent gas to obtain a three-phase mixture comprising a gas phase, an elemental bromine liquid phase, and an aqueous liquid phase.

23. The method of claim 22 further comprising separating said gas phase, said elemental bromine liquid phase, and said aqueous liquid phase from one another, wherein said elemental bromine liquid phase is essentially pure elemental bromine in a liquid state and comprises a first portion of said elemental bromine product.

24. The method of claim 22, wherein said gas phase includes oxygen and a first residual elemental bromine portion, the method further comprising recovering said first residual elemental bromine as a second portion of said elemental bromine product.

25. The method of claim 22, wherein said aqueous liquid phase includes water and a second residual elemental bromine portion dissolved therein, the method further comprising recovering said second residual elemental bromine as a third portion of said elemental bromine product.

26. A method for converting hydrogen bromide to elemental bromine comprising the steps of:
converting gaseous alkanes to liquid hydrocarbons in a gaseous alkane conversion process by brominating said gaseous alkanes and catalytically reacting resulting alkyl bromides to form said liquid hydrocarbons and a hydrogen bromide-containing gas;
deriving an initial hydrogen bromide-rich gas from said hydrogen bromide-containing gas;
thermally oxidizing a portion of said initial hydrogen bromide-rich gas at a thermal oxidation temperature to produce a first fraction of elemental bromine and a remainder of said initial hydrogen bromide-rich gas;
catalytically oxidizing at least a portion of said remainder of said initial hydrogen bromide-rich gas at a catalytic oxidation temperature to produce a second fraction of elemental bromine; and
recycling said first and second fractions of elemental bromine to said gaseous alkane conversion process to brominate said gaseous alkanes.

27. The method of claim 26, wherein said hydrogen bromide-containing gas has a lower hydrogen bromide concentration than said initial hydrogen bromide-rich gas.

28. The method of claim 26, wherein said initial hydrogen bromide-rich gas is said hydrogen bromide-containing gas.

29. The method of claim 26, wherein said hydrogen bromide-containing gas is a gaseous mixture containing hydrogen bromide and lower molecular weight hydrocarbons.
